(19) United States
(12) Patent Application Publication
(10) Pub. No.: US 2024/0023880 A1
(43) Pub. Date: Jan. 25, 2024

(54) WEARABLE MICROFLUIDIC BIOAFFINITY SENSOR FOR AUTOMATIC MOLECULAR ANALYSIS

(71) Applicant: California Institute of Technology, Pasadena, CA (US)

(72) Inventors: Wei GAO, PASADENA, CA (US); Jiaobing TU, PASADENA, CA (US)

(73) Assignee: California Institute of Technology, Pasadena, CA (US)

(21) Appl. No.: 18/225,403

(22) Filed: Jul. 24, 2023

Related U.S. Application Data
(60) Provisional application No. 63/391,669, filed on Jul. 22, 2022.
(60) Provisional application No. 63/521,418, filed on Jun. 16, 2023.

(51) Int. Cl. A61B 5/00; A61B 5/145

(52) U.S. Cl. CPC A61B 5/4266; A61B 5/14521; A61B 5/14539; A61B 5/14546

(57) ABSTRACT

Some implementations of the disclosure relate to a wearable biosensor device including an iontophoresis module configured to stimulate production of a sweat sample from skin of a user, the sweat sample including biomarkers; a microfluidic module configured to collect the sweat sample, mix the sweat sample with labeled detection reagents to obtain a mixture including the biomarkers bound to the labeled detection reagents, and route the mixture to a detection reservoir of the microfluidic module; and a sensor assembly including a bioaffinity sensor configured to quantify the biomarkers of the mixture in the detection reservoir to determine a concentration of the biomarkers present in the sweat sample. The bioaffinity sensor includes an electrode functionalized to bind to the biomarkers of the mixture. The bioaffinity sensor can quantify the biomarkers to determine their concentration with a sensitivity on the order nanomoles or picomoles.

### CROSS-REFERENCE TO RELATED APPLICATIONS

This application claims the benefit of U.S. Provisional Patent Application No. 63/391,669, filed Jul. 22, 2022, and titled “Wearable Microfluidic Bioaffinity Sensor For Automatic Molecular Analysis.” This application also claims the benefit of U.S. Provisional Patent Application No. 63/521,418, filed Jun. 16, 2023, and titled “Wearable Microfluidic Bioaffinity Sensor For Automatic Molecular Analysis.” All of the above applications are incorporated herein by reference in their entirety.

### TECHNICAL FIELD

The present disclosure relates generally to systems and methods for biomarker monitoring using a wearable biosensor device. Particular implementations are directed to automatic and non-invasive monitoring of protein or hormone biomarkers using a wearable microfluidic bioaffinity sensor that collects sweat samples.

### BACKGROUND

Recent advances in flexible electronics and digital health have transformed conventional laboratory tests into remote wearable molecular sensing that enables real-time monitoring of physiological biomarkers. Sweat contains abundant biochemical molecules ranging from electrolytes and metabolites, to large proteins, and importantly, it is readily accessible by non-invasive techniques. However, currently reported wearable biosensors are largely restricted to the detection of a limited selection of biomarkers such as electrolytes and metabolites at μM or greater concentrations via ion-selective and enzymatic sensors or direct oxidation/reduction. For example, the majority of clinically relevant protein biomarkers including C-reactive protein (CRP) are present at nM to pM levels in blood while the anticipated levels of proteins in sweat are expected to be much lower than in blood. Commercial point-of-care biomarker monitors are still bulky in size and cannot reach picomolar-level sensitivity to assess biomarker levels in non-invasively accessible alternative biofluids such as sweat and saliva.

### SUMMARY

The technology described herein relates to wearable bioaffinity sensor systems and methods capable of automatic and real-time monitoring of low levels of biomarkers such as hormone and protein biomarkers.

In one embodiment, a wearable biosensor device comprises: an iontophoresis module configured to stimulate production of a sweat sample from skin of a user, the sweat sample including biomarkers; a microfluidic module configured to collect the sweat sample, mix the sweat sample with labeled detection reagents to obtain a mixture including the biomarkers bound to the labeled detection reagents, and route the mixture to a detection reservoir of the microfluidic module; and a sensor assembly comprising a bioaffinity sensor configured to quantify the biomarkers of the mixture in the detection reservoir to determine a concentration of the biomarkers present in the sweat sample, the bioaffinity sensor comprising an electrode functionalized to bind to the biomarkers of the mixture.

In some implementations, the labeled detection reagents comprise first nanoparticles conjugated with detection antibodies that bind to the biomarkers; and a surface of the electrode comprises second nanoparticles conjugated with capture antibodies that bind to the biomarkers.

In some implementations, first nano particles and second nanoparticles are gold nanoparticles (AuNPs). In some implementations, the biomarkers comprise protein biomarkers or hormone biomarkers. In particular implementations, the biomarkers comprise CRP.

In some implementations, the wearable biosensor device is configured to quantify the biomarkers of the mixture to determine the concentration with a sensitivity of 1 micromole or less, 100 nanomoles or less, 10 nanomoles or less, 1 nanomole or less, 100 picomoles or less, or 10 picomoles or less.

In some implementations, the microfluidic module comprises: an inlet for collecting the sweat sample; a reagent reservoir including the labeled detection reagents, the reagent reservoir configured to refresh the sweat sample with the labeled detection reagents; a mixing channel for mixing the sweat sample refreshed with the labeled detection reagents to form the mixture including the labeled detection reagents bound to the biomarkers; the detection reservoir for receiving the mixture from the mixing channel; and an outlet for providing an outflow of the sweat sample from the detection reservoir.

In some implementations, the sensor assembly further comprises: a temperature sensor configured to measure a temperature of the skin; an ionic strength sensor configured to measure an ionic strength of the sweat sample; and/or a pH sensor configured to measure a pH level of the sweat sample. In some implementations, the wearable biosensor device is configured to calibrate readings from the bioaffinity sensor based on measurements made by the temperature sensor, the ionic strength sensor, and/or the pH sensor.

In some implementations, the sensor assembly comprises a multiplexed sensor array fabricated using laser-engraved graphene (LEG), the multiplexed sensor array including the bioaffinity sensor, the temperature sensor, the ionic strength sensor, and/or the pH sensor.

In some implementations, the wearable biosensor device comprises: a disposable patch including the iontophoresis module, the microfluidic module, and the sensor assembly, the disposable patch comprising an adhesive to directly adhere the disposable patch to the skin; and a flexible printed circuit board (FPCB) coupled to the patch, the FPCB configured to receive signals from the sensor assembly and power the wearable biosensor device.

In some implementations, the FPCB is reusable and configured to removably couple to the patch; and the FPCB comprises a processor configured to perform in situ signal processing of signals received from the sensor assembly, and a wireless communication module configured to wirelessly communicate, in real-time, with a mobile device.

In one embodiment, a method comprises: receiving, via an inlet of a microfluidic module, a sweat sample collected from skin, the sweat sample including protein or hormone biomarkers; reconstituting, within a reagent reservoir of the microfluidic module, the sweat sample with detection reagents configured to bind with the protein or hormone biomarkers, the detection regents comprising electroactive label molecules; binding, within a mixing channel of the microfluidic module, the detection reagents with the protein or hormone biomarkers to form a mixture including the protein or hormone biomarkers bound with the detection reagents; collecting, within a detection reservoir of the microfluidic module, the mixture of the protein or hormone biomarkers bound to the detection reagents, to bind the protein or hormone biomarkers to an electrode of a sensor assembly; refreshing the microfluidic module with one or more additional sweat samples not containing detection reagents to remove, via an outlet of the microfluidic module, unbound detection reagents; and estimating a concentration of the protein or hormone biomarkers present in the sweat sample by measuring an amount of the electroactive labels present at a surface of the electrode.

In some implementations, estimating the concentration of the protein or hormone biomarkers present in the sweat sample, comprises: estimating the concentration of the protein or hormone biomarkers with a sensitivity of 1 micromole or less, 100 nanomoles or less, 10 nanomoles or less, 1 nanomole or less, 100 picomoles or less, or 10 picomoles or less.

In some implementations, the method further comprises: obtaining, using one or more additional sensors of the sensor assembly, one or more additional biophysical sensor measurements comprising a temperature of the skin, a pH level of the sweat sample, or an ionic strength of the sweat sample; and calibrating, based on the one or more additional biophysical sensor measurements, the estimated concentration of the protein or hormone biomarkers.

In some implementations, the method further comprises: prior to receiving the sweat sample via the inlet, inducing, using an iontophoresis module in contact with the skin, the sweat sample.

In some implementations, the protein biomarkers are CRP. In some implementations, the detection reagents further comprise first nanoparticles conjugated with detection antibodies that bind to the CRP; and a surface of the electrode comprises second nanoparticles conjugated with capture antibodies that bind to the CRP.

In some implementations, the first nanoparticles and second nanoparticles are gold nanoparticles; and the electroactive label molecules are redox molecules.

In one embodiment, a method comprises: adhering, to skin of a user, a patch that includes a microfluidic module and sensor assembly; collecting, in the microfluidic module, a sweat sample obtained from the skin; mixing, within the microfluidic module, the sweat sample with reagents to obtain a mixture that comprises the reagents bound to protein or hormone biomarkers contained in the sweat sample; and estimating, from the mixture, using the sensor assembly, a concentration of the protein or hormone biomarkers in the sweat sample.

In some implementations, the method further comprises: monitoring, in real-time, based on the concentration of the protein or hormone biomarkers estimated using the sensor assembly, a health condition of the user.

In some implementations, monitoring in real-time, the health condition of the user, comprises: comparing the concentration of the protein or hormone biomarkers estimated using the sensor assembly to a threshold to determine a biological condition of the user. For example, the concentration of CRP or some other inflammatory biomarker that was estimated using the sensor assembly can be compared to a threshold to determine whether the user is presently experiencing an inflammatory response.

In some implementations, the health condition comprises: heart disease, chronic obstructive pulmonary disease, inflammatory bowel disease, an active infection, or a past infection.

In some implementations, the method further comprises: presenting to the user, in real-time, via a mobile device communicatively coupled to the patch via a wireless communication medium, the concentration of the protein or hormone biomarkers estimated using the sensor assembly.

Other features and aspects of the disclosed technology will become apparent from the following detailed description, taken in conjunction with the accompanying drawings, which illustrate, by way of example, the features in accordance with implementations of the disclosed technology. The summary is not intended to limit the scope of any inventions described herein, which are defined by the claims and equivalents.

### BRIEF DESCRIPTION OF THE DRAWINGS

The present disclosure, in accordance with one or more implementations, is described in detail with reference to the following figures. The figures are provided for purposes of illustration only and merely depict example implementations. Furthermore, it should be noted that for clarity and ease of illustration, the elements in the figures have not necessarily been drawn to scale.

FIG. 1A shows an environment for using a wearable biosensor device including a sweat sensor patch for automatic and non-invasive biomarker monitoring, in accordance with some implementations of the disclosure.

FIG. 1B shows a cross-sectional view of the sweat sensor patch of FIG. 1A in operation and adhered to skin, in accordance with some implementations of the disclosure.

FIG. 1C shows an optical image of a sensor patch, in accordance with some implementations of the disclosure.

FIG. 1D shows an optical image of a vertical stack assembly of a fully integrated biosensor device including a sensor patch and a FPCB, in accordance with some implementations of the disclosure.

FIG. 1E shows an exploded view of a wearable biosensor device, in accordance with some implementations of the disclosure.

FIG. 2 is a flow diagram illustrating an example method of assembling a sweat sensor patch, in accordance with some implementations of the disclosure.

FIG. 3 illustrates that can be used during assembly of a microfluidic module, in accordance with some implementations of the disclosure.

FIG. 4 illustrates components of a microfluidic module and sensor assembly that can be utilized during automatic bioaffinity sensing, in accordance with some implementations of the disclosure.

FIG. 5 is an operational flow diagram illustrating example operations performed during automatic bioaffinity sensing, using the components of biosensor device illustrated in FIG. 4, in accordance with some implementations of the disclosure.

FIG. 6A illustrates a particular implementation for realizing automatic wearable CRP detection in situ using labeled CRP detector antibody(dAb)-conjugated AuNPs.

FIG. 6A illustrates reconstitution and incubation operations within a microfluidic module of a wearable bioaffinity sensor, in accordance with a particular implementation of the disclosure.

FIG. 6B illustrates refreshment and detection operations within a microfluidic module of a wearable bioaffinity sensor, in accordance with a particular implementation of the disclosure.

FIG. 6C illustrates a detection operation performed by a wearable bioaffinity sensor, in accordance with a particular implementation of the disclosure.

FIG. 7 shows an enlarged view of a working electrode surface conceptually illustrating the binding process at the surface of a working electrode between capture antibodies on the electrode surface and biomarkers bound to detection antibodies received via a microfluidic module, in accordance with some implementations of the disclosure.

FIG. 8 illustrates an enlarged plan view of the electronics of a FPCB of a wearable biosensor device, in accordance with some implementations of the disclosure.

FIG. 9 is a block diagram illustrating an example electronic system of a biosensor device used for protein or hormone biomarker sensing, in accordance with some implementations of the disclosure.

FIG. 10 illustrates an example graphical user interface (GUI) that can be presented to a user by running a mobile application used in conjunction with a wearable biosensor device for noninvasive automatic biomarker monitoring, in accordance with some implementations of the disclosure.

FIG. 11 shows scanning electron microscope (SEM) images of raster-mode engraved graphene of LEG electrodes for CRP sensing, LEG-AuNPs of the LEG electrodes for CRP sensing, vector-mode engraved LEG electrodes for pH sensing, and vector-mode engraved electrodes for temperature sensing, in accordance with one particular implementation.

FIG. 12A illustrates a schematic of layers of a functionalized LEG-AuNPs working electrode of a bioaffinity sensor, in accordance with a particular implementation of the disclosure.

FIG. 12B illustrates a surface functionalization process of an LEG-AuNPs working electrode of a bioaffinity sensor, in accordance with a particular implementation of the disclosure.

FIG. 12C shows an SEM image of a mesoporous LEG electrode, in accordance with a particular implementation of the disclosure.

FIG. 12D shows a transmission electron microscopy (TEM) image of AuNP-decorated graphene flakes, in accordance with a particular implementation of the disclosure.

FIG. 12E illustrates amperometric responses and SEM images of CRP sensors based on LEG modified with poly(pyrrolepropionic acid) (PPA) and pyrenebutyric acid (PBA).

FIG. 12F illustrates amperometric responses of CRP sensors based on AuNPs/self-assembled monolayer (SAM) and laser-engraved graphene oxide by electrochemical oxidation (LEGO), as well as a plot illustrating a sensor performance comparison of different functionalization methods.

FIG. 12G shows batch to batch variations in electrochemical performance of LEG electrodes and LEG-AuNPs electrodes in accordance with some implementations of the disclosure.

FIG. 12G, includes plots showing oxidation peak heights in the cyclic voltammograms (CVs) of LEG electrodes and LEG-AuNPs electrodes in accordance with some implementations of the disclosure.

FIG. 12H includes plots showing a comparison of the electrochemical performances of redox probe conjugated dAb and dAb-conjugated AuNP.

FIG. 12I is a TEM image showing dispersed dAb-loaded AuNPs with protein corona shells.

FIG. 12J shows square wave voltammetry (SWV) voltammograms of CRP sensors in accordance with a particular implementation of the disclosure.

FIG. 12K shows the corresponding calibration plot of the CRP sensors of FIG. 12J.

FIG. 12L is a plot illustrating the selectivity of a CRP sensor to potential interferences in sweat.

FIG. 12M is another plot illustrating the selectivity of a CRP sensor to potential interferences in sweat.

FIG. 12N is a plot showing validation of a CRP sensor in human sweat samples and saliva samples, in accordance with a particular implementation of the disclosure.

FIG. 13A depicts a high level schematic of the evaluation of sweat CRP for the non-invasive monitoring of various health conditions that could be associated with elevated CRP in healthy or patient populations, in accordance with some implementations of the disclosure.

FIG. 13B shows a box-and-whisker plot of a study of CRP levels in iontophoresis-extracted sweat and serum samples from patients with chronic obstructive pulmonary disease (COPD) and without COPD, in accordance with some implementations of the disclosure.

FIG. 13C shows a box-and-whisker plot of a study of CRP levels in sweat and serum samples from healthy participants, patients with heart failure with reduced ejection fraction (HFrEF), and patients with heart failure with preserved ejection fraction (HFPEF), in accordance with some implementations of the disclosure.

FIG. 13D shows a box-and-whisker plot of a study of CRP levels in sweat and serum samples from three patients with active infection on two consequent days, in accordance with some implementations of the disclosure.

FIG. 13E is a plot showing a computed correlation of serum and sweat CRP levels.

FIG. 14A includes plots showing on-body multiplexed physicochemical analysis and CRP analysis with real-time sensor calibrations of healthy never smokers using a wearable sensor in accordance with some implementations of the disclosure.

FIG. 14B includes plots showing on-body multiplexed physicochemical analysis and CRP analysis with real-time sensor calibrations of healthy smokers using a wearable sensor in accordance with some implementations of the disclosure.

FIG. 14C includes plots showing on-body multiplexed physicochemical analysis and CRP analysis with real-time sensor calibrations of a patient with COPD using a wearable sensor in accordance with some implementations of the disclosure.

FIG. 14D includes plots showing on-body multiplexed physicochemical analysis and CRP analysis with real-time sensor calibrations of participants who previously had COVID-19 using a wearable sensor in accordance with some implementations of the disclosure.

15A includes a plot showing the measured admittance response of an impedimetric ionic strength sensor in NaCl solutions.

FIG. 15B includes a calibration plot of the impedimetric ionic strength sensor associated with FIG. 15A

FIG. 15C includes a plot shows simulated CRP-dAb concentration changes on a working electrode over time.

FIG. 15D shows simulated CRP-dAb concentrations showing phases of automatic sweat sampling and reagents routing toward in situ CRP detection.

FIG. 15E includes a plot showing admittance changes of an LEG ionic strength sensor as a function of time during four stages of automatic CRP sensing process in a laboratory flow test using artificial sweat.

FIG. 15F includes a plot showing admittance responses of an LEG ionic strength sensor as a function of time at different flow rates in a laboratory flow test using artificial sweat.

FIG. 15G includes an admittance plot showing the influence of flow rates on microfluidic automatic CRP sensing.

FIG. 15H includes a voltammogram plot showing the influence of flow rates on microfluidic automatic CRP sensing.

FIG. 15I includes an admittance plot showing the influence of ionic strengths on microfluidic automatic CRP sensing.

FIG. 15J includes a voltammogram plot showing the influence of ionic strengths on microfluidic automatic CRP sensing.

The figures are not exhaustive and do not limit the present disclosure to the precise form disclosed.

### DETAILED DESCRIPTION

Despite recent efforts in the development of wearable bioaffinity biosensors for trace-level biomarkers such as cortisol, the accurate and in situ detection of biomarkers such as sweat protein or hormone biomarkers remains a major challenge due to their extremely low concentrations (e.g., nM or pM levels) and the large interpersonal and intrapersonal variations in sweat compositions. For example, the detection of protein biomarkers usually requires integrating bioaffinity receptors such as antibodies and aptamers. However, such techniques typically require lengthy target incubation, labor-intensive washing steps, and the addition of redox solutions for signal transduction. In addition, the current turnaround time (1 day or more) of high-sensitivity clinical biomarker tests such as the high-sensitivity CRP Test (hsCRP) may not meet the need for frequent assessments. For example, in addition to hospitalized cases that require close monitoring of inflammatory state, many chronic diseases, such as COPD and inflammatory bowel disease, could benefit from at-home, daily or frequent, fully automatic, and non-invasive assessment of CRP for disease management.

As such, there is a need for a wearable biosensing technology that allows automatic in situ monitoring of ultra-low-level circulating biomarkers at home and in community settings. To this end, some implementations of disclosure are directed to systems and methods for wearable and real-time electrochemical detection of low-concentration protein and hormone biomarkers such as inflammatory biomarkers in sweat. In accordance with some implementations of the disclosure, a biosensor device for biomarker sampling can include: an iontophoresis module that stimulates production of sweat, a microfluidic module for sweat sampling and for labeled reagent routing and replacement, and an electrochemical bioaffinity sensor (including, but not limited to an immunosensor, DNA sensor, and aptamer sensor) for quantifying a biomarker contained in the sweat. Particular implementations are directed to a wearable and wireless patch that includes the aforementioned components for the real-time electrochemical detection of low level concentrations of biomarkers in sweat. During on-body operation, the patch can conformally adhere to the skin through medical adhesive with in situ biomarker sensing performed in the microfluidics without direct sensor-skin contact. In a particular embodiment, the inflammatory biomarker CRP can be monitored in sweat samples.

In accordance with some particular implementations, the biosensor device can utilize a bioaffinity sensor (e.g., CRP sensor) for quantifying the biomarker (e.g., CRP) via an electrode functionalized with nanoparticle-conjugated capture antibodies (e.g., anti-CRP capture antibodies). In accordance some particular implementations, the bioaffinity sensor can be part of a graphene-based sensor array that also includes sensors for ionic strength, pH, and/or temperature measurements, for the real-calibration of the bioaffinity sensor.

Various benefits can be realized by implementing the systems and methods described herein. First the wearable biosensor device described herein can enable real-time, non-invasive, and wireless biomarker analysis in both healthy and patient populations. This could facilitate the management and/or detection of chronic diseases by providing real-time sensitive analysis of biomarkers present in sweat of a user. Second, by virtue of combining particular nanomaterials and chemistry techniques (e.g., a combination of capture receptor such as antibody immobilized mesoporous graphene-Au nanoparticles for efficient target recognition and thionine-tagged detector antibody-conjugated Au nanoparticles for signal transduction and amplification), the technology described herein could realize sweat CRP or other biomarker analysis with high sensitivity, selectivity, and efficiency. For example, in contrast to previous wearable technologies for monitoring biomarkers previously reported LEG-based sensors that detect metabolites at μM or higher level, the technology described herein could be used to realize highly sensitive detection of ultra-low-level biomarkers in situ with a 6 orders-of-magnitude (e.g., picomolar level) improvement in sensitivity.

Third, biosensor device modules described herein can enable autonomous sweat induction, sampling, reagent routing, and fully automatic bioaffinity sensing in situ on the skin of a user. Further, by virtue of utilizing multiple sensor modalities in some implementations, the influence of interpersonal variations on wearable sensing can be mitigated and allow real-time biomarker data calibration. These additional sensor modalities could also be used to provide a more comprehensive assessment of the physiological status.

Further still, utilizing the technology described herein to perform experiments involving measurement of CRP levels in patients, the presence of CRP was confirmed in human sweat from healthy subjects, and elevated CRP levels were discovered in sweat from patients with various chronic and acute inflammations associated with health conditions including heart failure, COPD, and active and past infections (e.g., COVID-19). Moreover, by virtue of utilizing the technology described herein to perform experiments involving measurement of CRP levels in patients, a strong correlation between sweat and blood serum CRP levels was discovered in both healthy and patient populations, indicating the utility of the technology described herein in non-invasive disease classification, monitoring, and/or management.

These and other benefits realized by implementing the technology described herein are further describe below.

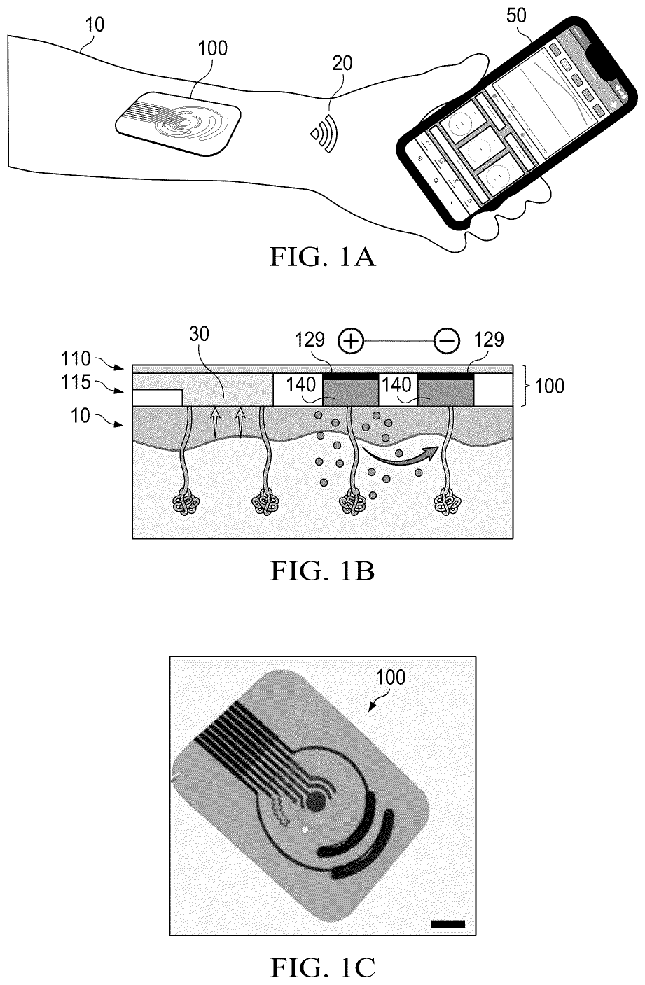

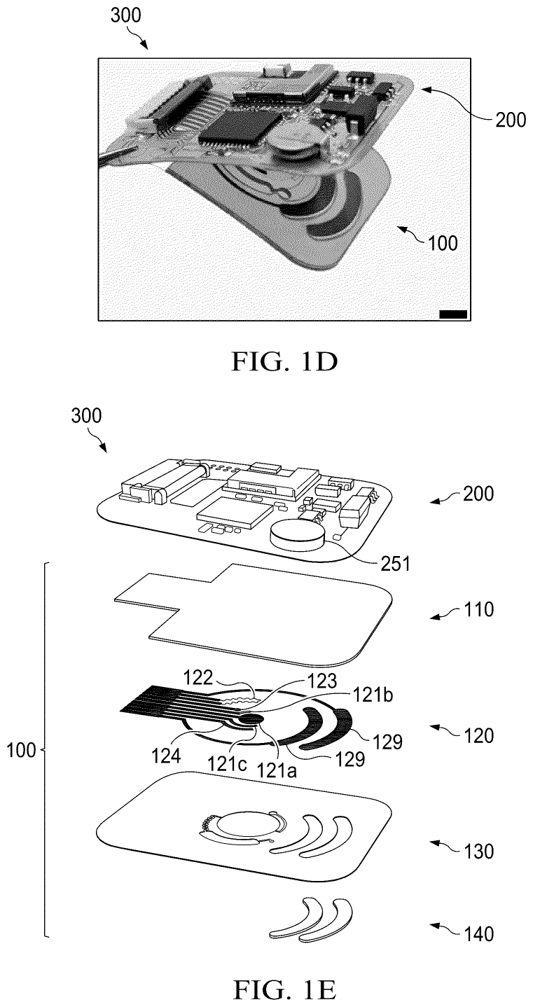

FIGS. 1A-1E illustrate an example biosensor device 300 including a sweat sensor patch 100, and an environment for using the biosensor device 300, in accordance with some implementations of the disclosure. As depicted by the example of FIGS. 1A-1B, the sweat sensor patch 100 of the biosensor device 300 can be adhered to the skin 10 of a user (e.g., a human patient). As depicted by FIG. 1B, which shows a cross-sectional view of the sweat sensor patch 100 in operation and adhered to skin 10, the sweat sensor patch 100 can include a backing layer/substrate 110 and one or more layers 115 including a medical adhesive (e.g., medical tape) used to directly attach the sensor patch 100 to skin 100. Iontophoresis electrodes 129 can interface the skin 100 with a layer of hydrogel agent 140 applied in between to stimulate the production of sweat 30. The hydrogel agent 140, which can be a component of sensor patch 100, can be an agarose gel containing carbachol (carbagel). An electric current can travel to the electrodes 129, which enable the transdermal transport of carbachol to the sweat glands, triggering the flow of the sweat stimulating agent into the skin 10, and stimulating the production of sweat 30 as needed. Considering that the potential users of the technology can include sedentary and immobile patients, an iontophoresis module, including the pair of electrodes, can provide the benefit of on-demand delivery of a hydrogel agent (e.g., cholinergic agonist carbachol from the carbagel) for autonomous sweat stimulation throughout daily activities without the need for vigorous exercise.

During operation, the biosensor device 300 is configured to collect biophysical data corresponding to the user, including data associated with biomarkers collected from the user's sweat 30, and communicate the data to a mobile device 50 via a wireless communication link 20. The wireless communication link 20 can be a radio frequency link such as a Bluetooth® or Bluetooth® low energy (LE) link, a Wi-Fi® link, a ZigBee link, or some other suitable wireless communication link. In some embodiments, a low energy and/or short-range wireless communication link can preferably be used for data transfer. The mobile device 50 can be a smartphone, a smartwatch, a head mounted display (HMD), or other suitable mobile device that can run an application that displays health information (e.g., inflammatory biomarker data, temperature data, etc.) associated with the data received from the biosensor device 300. In some implementations, the application can analyze and/or organize data collected from the biosensor device 300.

FIG. 1C shows an optical image of a sensor patch 100 in accordance with some implementations of the disclosure. The imaged sensor patch in this example is a disposable microfluidic graphene sensor patch. FIG. 1D shows an optical image of a vertical stack assembly of the fully integrated biosensor device 300 including the sensor patch 100 shown in FIG. 1C and a FPCB 200. In both optical images, the scale bars 0.5 cm.

FIG. 1E shows an exploded view of a biosensor device 300, in accordance with some implementations of the disclosure. The biosensor device 300 includes sweat sensor patch 100 and FPCB 200. The sweat sensor patch 100 includes backing substrate 110, sensor assembly 120, microfluidic layer/module 130, and hydrogel agent 140. The backing substrate 110 can be made of a polyimide film or other suitable material, particularly materials that are lightweight, flexible, heat resistant, and/or chemical resistant. For example, the microfluidic biosensor patch can be fabricated on a polyimide substrate via CO₂ laser engraving.

As shown, the sensor assembly 120 can include a bioaffinity sensor 121a-121c as well as additional sensors 122-124. The bioaffinity sensor 121a-121c can include a working electrode 121a including a coating that selectively binds to the biomarker of interest present in a sweat sample, a reference electrode 121b, and a counter electrode 121c for sweat biomarker capturing and electrochemical analysis. In a particular embodiment, the bioaffinity sensor 121a-121c is an inflammatory biomarker sensor (e.g., a CRP sensor) that binds to an inflammatory biomarker of interest (e.g., CRP). In some implementations, the working electrode 121a can be coated with nanoparticles conjugated with antibodies that bind to the biomarker of interest. In particular implementations, the working electrode 121a is functionalized with AuNPs conjugated with capture antibodies (cAbs). For example, the cAbs can be anti-CRP cAbs. The AuNP can be electrodeposited. In particular implementations, the reference electrode 121b is an Ag/AgCl reference electrode. The aforementioned design can enable highly sensitive and efficient electrochemical detection of trace-level sweat biomarkers such as hormones or proteins, including CRP, in situ on the skin. For example, in some implementations, the sensor assembly 120 including bioaffinity sensor 121a-121c is configured to determine the concentration of the biomarkers with a sensitivity of 1 micromole or less, 100 nanomoles or less, 10 nanomoles or less, 1 nanomole or less, 100 picomoles or less, or even 10 picomoles or less. Other nanoparticles that can be conjugated with an antibody that binds to the biomarker can include iron oxide nanoparticles, quantum dots, silver nanoparticles, copper nanoparticles, copper oxide nanoparticles, etc.

The additional sensors can include a temperature sensor 122, a pH sensor 123, and an ionic strength sensor 124. In one implementation, temperature sensor 122 is a strain-insensitive temperature sensor. In one implementation, pH sensor is a potentiometric sweat pH sensor. In one implementation, ionic strength sensor is an impedimetric ionic strength sensor. As further described below, having additional, integrated pH, temperature, and ionic strength sensors can enable real-time personalized biomarker data calibration to mitigate the interpersonal sample matrix variation-induced sensing error, and provide a more comprehensive assessment of the physiological status. In some implementations, the combination of sensors, including bioaffinity sensor 121a-121c and sensors 122-124 can be implemented as a multiplexed sensor array. In other implementations, some of the additional sensors can be excluded, or other additional sensors can be included to enable calibration.

The sensor assembly 120, including electrodes 129, bioaffinity sensor 121a-121c, and sensors 122-124, can be formed as LEG sensor assembly. LEG fabrication may enable large scale production of biosensor systems, via CO2 laser engraving, at relatively low cost. An LEG sensor can be advantageous because it can be printed using a modified conventional printer. Printable wearable sensor patches can be fabricated on a large scale at a relatively low cost. This may allow for disposable sensor patches which may be worn by an individual for an extended of time (e.g., 12-24 hrs), which can be replaced on a daily level, and which can collect health information without invasive testing and the need for a human patient to come in to a physical laboratory for repeated testing.

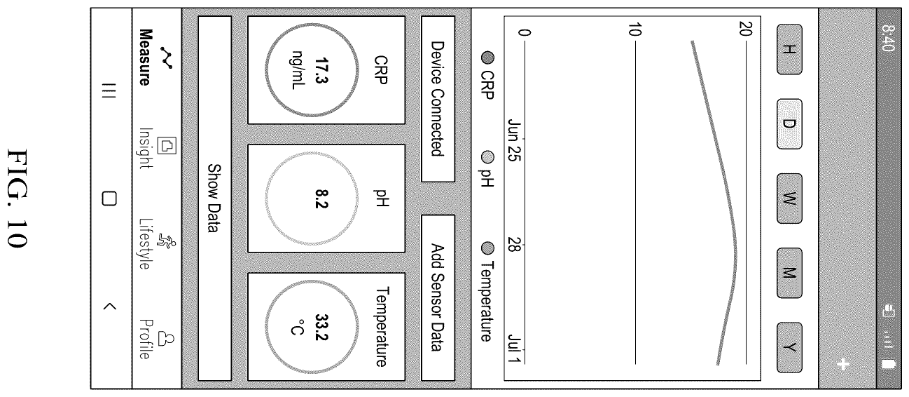

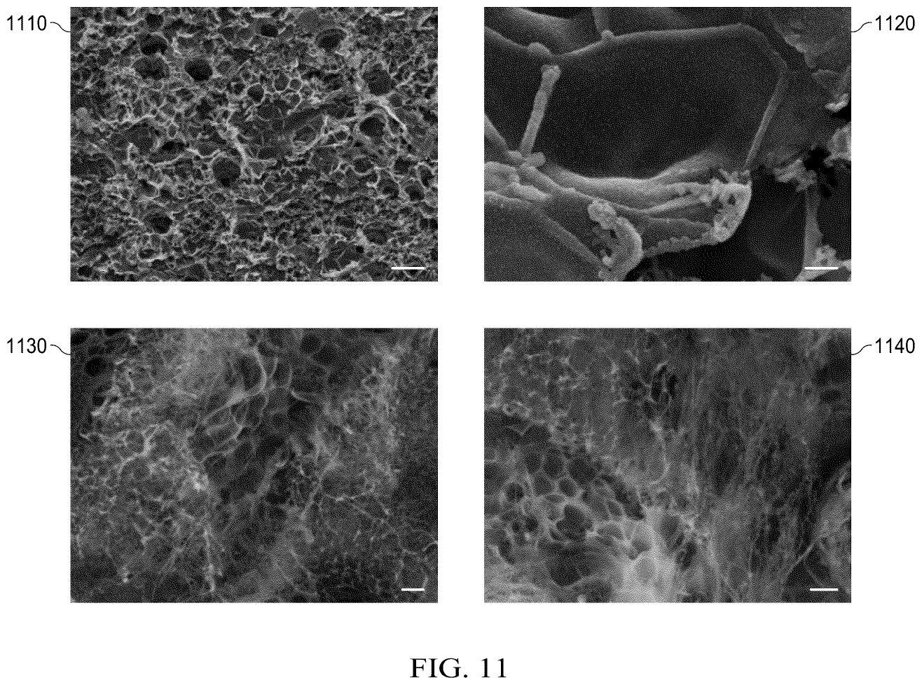

FIG. 11 shows SEM images of raster-mode engraved graphene of LEG electrodes for CRP sensing (image 1110), LEG-AuNPs of the LEG electrodes for CRP sensing (image 1120), vector-mode engraved LEG electrodes for pH sensing (image 1130), and vector-mode engraved electrodes for temperature sensing (image 1140), in accordance with one particular implementation. The scale bars for images 1110-1120 are 10 μm and 1 μm. The scale bars for image 1130-1140 are 2 μm.

The FPCB 200 can be configured for iontophoretic sweat induction, sensor data acquisition and/or wireless communication with a mobile device 50. During assembly, the FPCB 200 can interface on top of the patch 100 to form the fully integrated wearable biosensor device 300. The FPCB 200 can be configured as a reusable electronic system that interfaces with disposable, point-of-care sensor patches 100. A battery 251 (e.g., lithium battery) can power the system, enabling functions such as wireless communication. In other implementations, the biosensor device 300 can be powered by other or additional means such as by human motion, by a small solar panel, and/or by a biofluid powering system that powers the device using collected sweat flow.

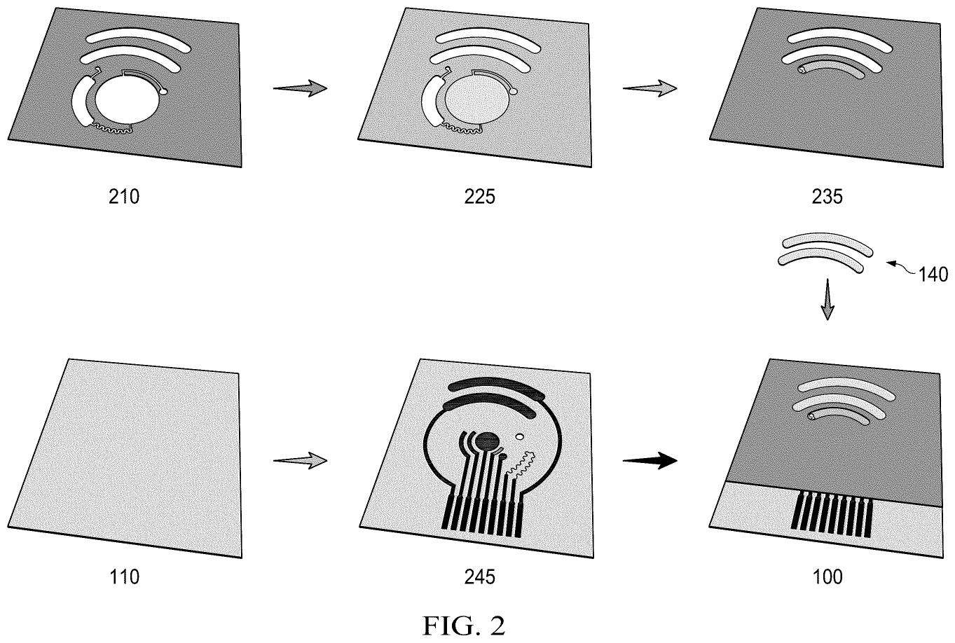

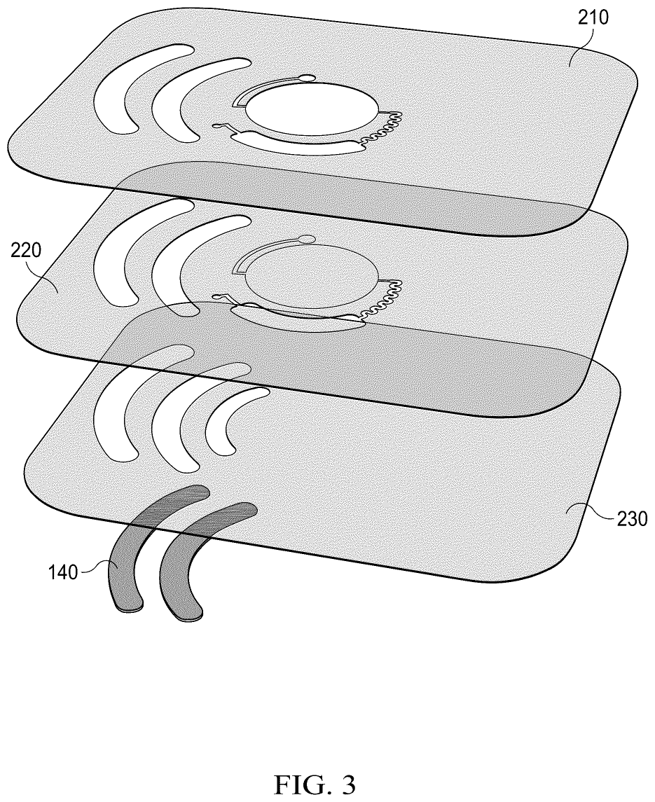

FIG. 2 is a flow diagram illustrating an example method of assembling a sweat sensor patch 100, in accordance with some implementations of the disclosure. FIG. 2 will be described with FIG. 3, which illustrates layers 210-230 that can be used during assembly of a microfluidic module 130. A microfluidic module 130 that is flexible can be assembled by stacking laser-cut layers 210-230. Cutouts can be formed in layers 210-230 for one or more inlets, a reagent reservoir, a mixing channel, a detection reservoir, one or more outlets, one or more channels, hydrogel, and/or other components of the sweat sensor patch 100. In this particular example, layer 210 is configured as a reservoir layer 210, layer 220 is configured as an inlet layer 220, and layer 230 is configured as a collection layer 230. The collection layer 230 can be patterned with one or more wells to collect sweat. The inlet layer 220 can include one or more inlets and/or channels via which the sweat flows through. The reservoir layer 210 can include a reservoir that receives the sweat flowing through the inlets and/or channels, and an outlet via which the sweat may flow through after sampling.

Each of the reservoir layer 210 and collection layer 230 can be a patterned medical adhesive such as medical tape that can be double-sided. The inlet layer 220 can be formed of a thermoplastic polymer resin such as Polyethylene terephthalate (PET). As depicted, the inlet layer 220 can be stacked/adhered over the reservoir layer 210 to form assembly 225. The collection layer 230 can be stacked/adhered over the assembly 225 to form an assembly 235 corresponding to the microfluidic module 130.

Also depicted in FIG. 2 is a backing layer 110 (e.g., polyimide layer) on which sensor assembly 120 can be printed or otherwise deposited to form assembly 245. During further assembly of sensor patch 100, the hydrogel agent 140 can be applied to assembly 235, and the assembly 235 can be stacked/adhered over the assembly 245.

In some implementations, the biosensor device 300 can be designed to have good mechanical flexibility and stability toward practical usage during physical activities. For example, each individual sensor could be designed such that it shows minimal variations under a moderate radius of bending curvature (e.g., 5 cm). In addition, more strain-insensitive sensor designs could be included as needed.

It should be appreciated that other methods of assembly are contemplated other than the one illustrated in FIG. 2, and that other biosensor assemblies besides wearable patches are contemplated as being in accordance with the technology described herein. For instance, the components of the biosensor device 300, including one or more of the components of the FPCB 200 and sensor patch 100 could instead be integrated into a wearable device such as a smartwatch or HMD. For example, components of the FPCB 200 and sweat sensor patch 100 could be incorporated into an area of a smartwatch that contacts a user's skin. In this example, the smartwatch could itself run an application that displays health information associated with the collected data, and/or alternatively communicate the data to another mobile device 50 such as a smartphone or wearable HMD that runs an application as described above.

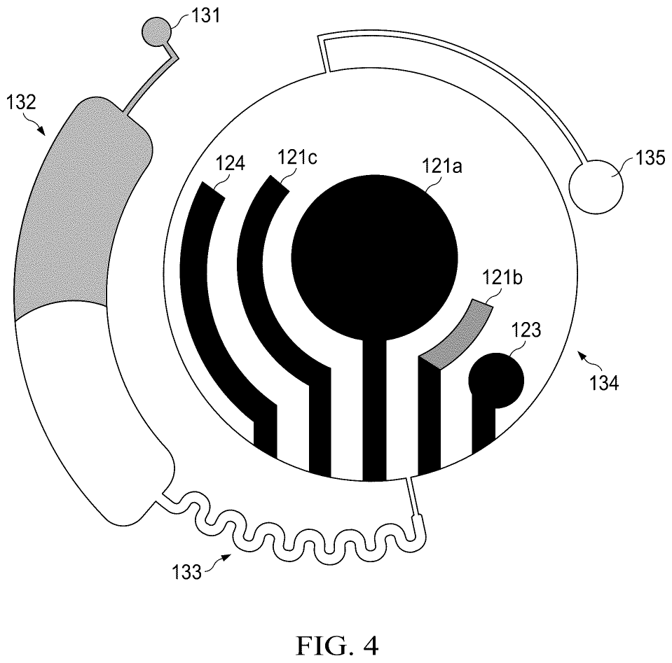

FIG. 4 illustrates components of a microfluidic module 130 and sensor assembly 120 that can be utilized during automatic bioaffinity sensing, in accordance with some implementations of the disclosure. As depicted, the microfluidic module 130 can include various fluidically coupled components, including an inlet 131 for receiving a sweat sample, a reagent reservoir 132 including detection reagents, a mixing channel 133, a detection reservoir 134 for capture and quantification of sweat biomarkers, and an outlet 135 that provides a channel for an outflow of the sweat sample. As described above, the sensor assembly 120 can include pH sensor 123, ionic strength sensor 124, and an biosensor including working electrode 121a, reference electrode 121b, and counter electrode 121c.

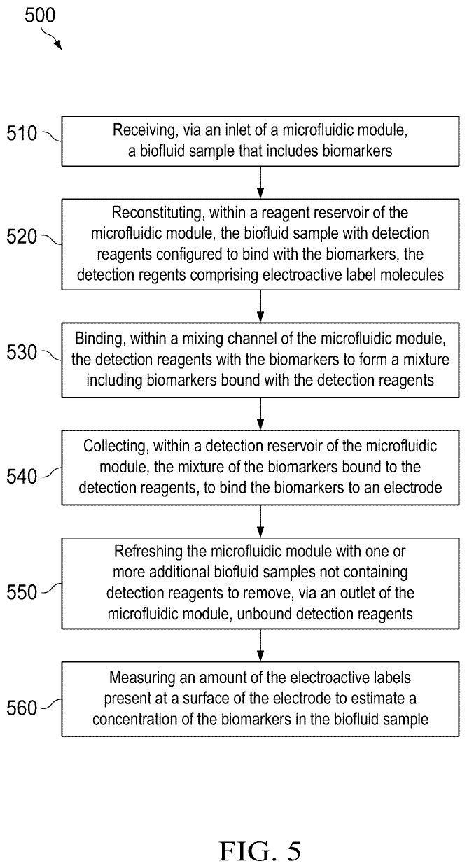

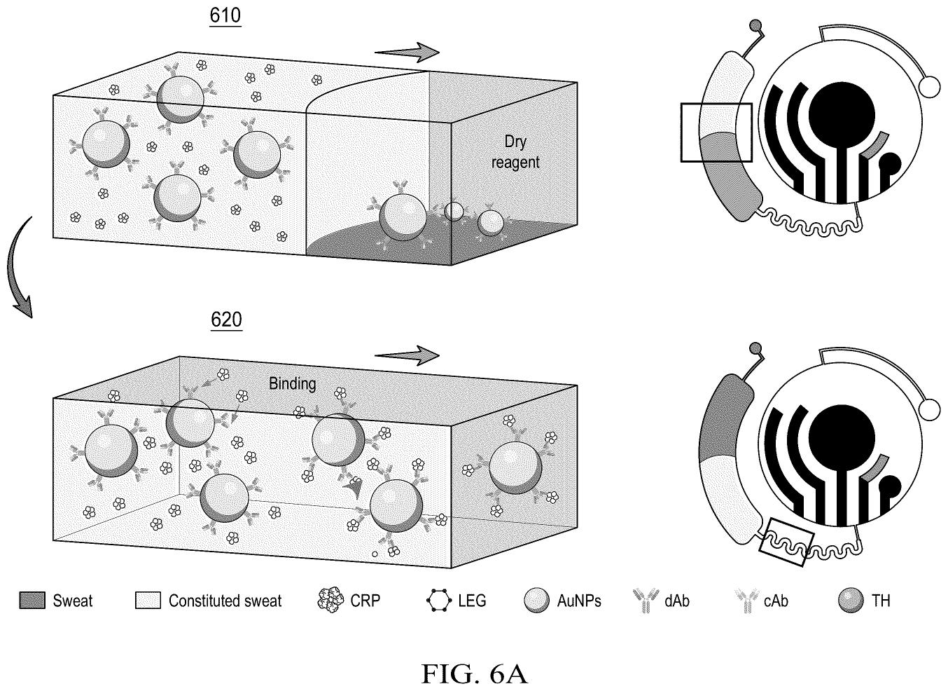

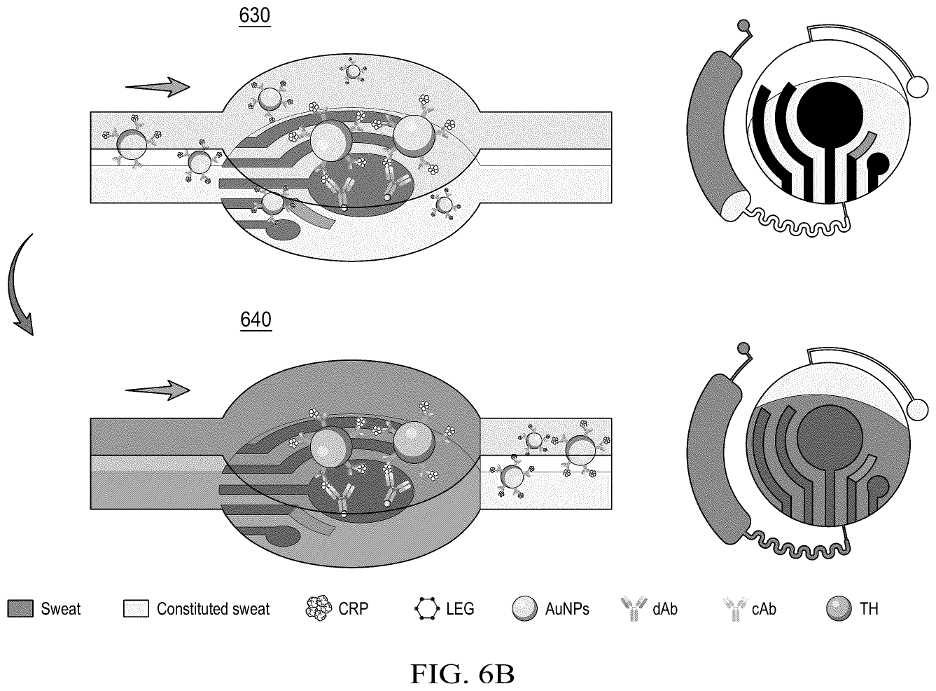

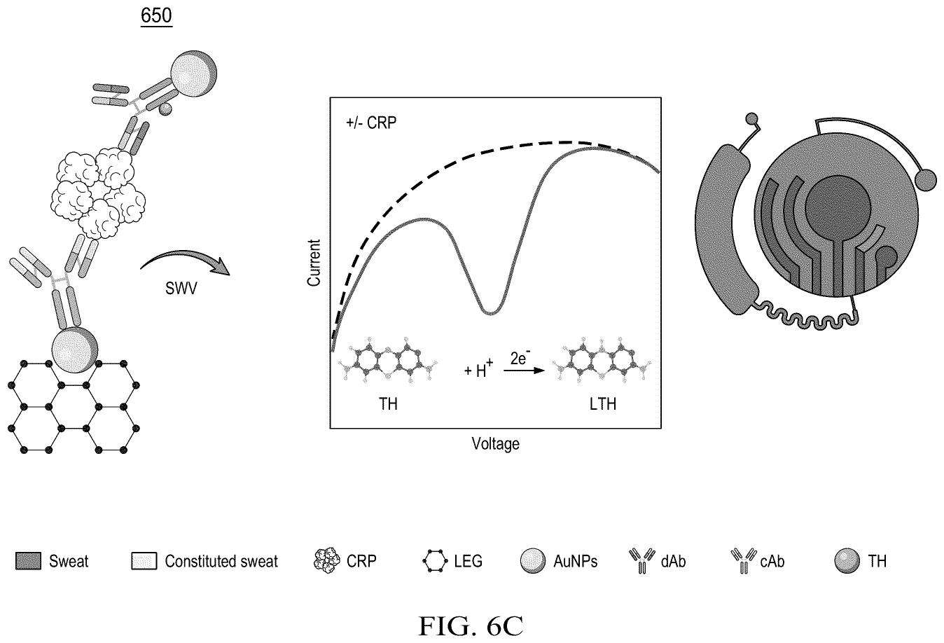

FIG. 5 is an operational flow diagram illustrating example operations performed during automatic bioaffinity sensing, using the components of biosensor device 300 illustrated in FIG. 4, in accordance with some implementations of the disclosure. FIG. 5 will be described with reference to FIGS. 6A-6C, which illustrate a particular implementation for realizing automatic wearable CRP detection in situ using labeled CRP dAb-conjugated AuNPs. However, it should be appreciated that the biosensor device 300 described herein can be configured to realize automatic detection in sweat of other biomarkers besides CRP, especially biomarkers that could be present in low (e.g., picomolar or nanomolar) concentrations, including hormones, proteins, peptides, and the like.

Operation 510 includes receiving, via an inlet 131, a biofluid sample that includes biomarkers. The biofluid sample can be a sweat sample that is autonomously induced using an iontophoresis module as described above (e.g., using electrodes 129 and carbagel 140), and it can flow into the microfluidic module 130 via inlet 131.

Operation 520 includes, reconstituting, within the reagent reservoir 132, the biofluid sample with detection reagents configured to bind with biomarkers contained in the biofluid, the detection regents comprising electroactive label molecules. The detection reagents can be deposited in the reagent reservoir 132 prior to biofluid collection. As the biofluid enters the reagent reservoir 132, it carries away the deposited detection reagents. For example, FIG. 6A illustrates reconstitution 610 within a reagent reservoir that stores labeled CRP dAbs-conjugated AuNPs. An electroactive redox molecule such as thionine (TH) can be used to label the nanoparticle conjugates to achieve direct electrochemical sensing. The nanoparticles conjugated with the electroactive redox molecules and dAbs can enable efficient electrochemical signal transduction (Signal ON) and signal amplification.

Operation 530 includes, binding, within the mixing channel 133, the detection reagents with the biomarkers contained in the biofluid sample to form a mixture. FIG. 6A illustrates binding 620 of detection reagents including AuNPs conjugated with CRP dAbs and redox molecule TH within a mixing channel. In the illustrated examples, the mixing channel 133 has a serpentine shape that can facilitate binding and control the amount of binding time. For example, the serpentine shape can facilitate dynamic binding between CRP and dAb. In other implementations, the mixing channel 133 can comprise a different shape.

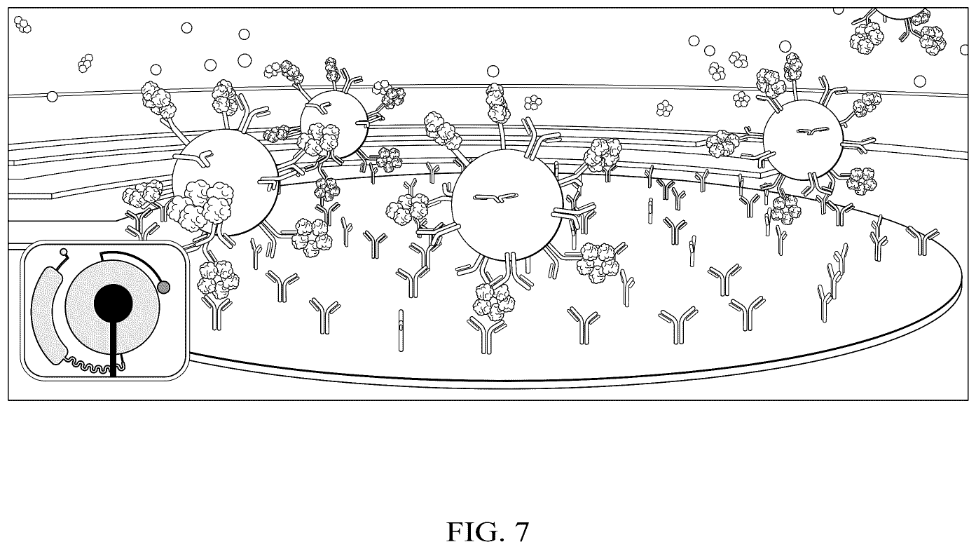

Operation 540 includes, collecting, within the detection reservoir 134, the mixture from the mixing channel 133 to bind the biomarkers, previously bound to the labeled detection reagents, to the working electrode 121a. For example, FIG. 6B illustrates an incubation process 630 via which CRP-dAb is allowed to bind with an anti-CRP cAb functionalized LEG-AuNPs working electrode. As the mixture enters the detection reservoir 134 from mixing channel 133, it can slowly fill the chamber before exiting the outlet 135. The size of the detection reservoir 134 can be optimized to allow sufficient time for binding with the working electrode 121a to take place. By way of further example, FIG. 7, shows an enlarged view of a working electrode surface conceptually illustrating the binding process that can take place at the surface of a working electrode between capture antibodies on the electrode surface and biomarkers bound to detection antibodies received via a microfluidic module. For simplicity, the AuNPs of the working electrode are not shown in this example.

Operation 550 includes, refreshing the microfluidic module 130 with one or more additional biofluid samples not containing detection reagents to remove unbound detection reagents from detection reservoir 134 via outlet 135. For example, a fresh sweat stream can continue to enter and refresh the microfluidics to remove unbound detection reagents and achieve removal of passive labels prior to detection. By way of example, FIG. 6B illustrates a refreshment operation 640 via which the detection reagent mixture that is unbound is removed. By virtue of performing the refreshment operation, the quantification of biomarkers contained in the sweat sample can be improved.

Operation 560 includes measuring an amount of electroactive label present at the working electrode surface to estimate a concentration of the biomarker. Any one of a number of voltammetric techniques that correlate current to concentration can be applied to make the measurement of the amount of electroactive label bound at the electrode surface. For example, differential pulse voltammetry (DPV), SWV, linear sweep voltammetry (LSV), or some other voltammetric technique can be used to make the measurement. It should be noted that because the electroactive label molecules are directly conjugated to the detection reagents, their amount can be directly correlated to the amount of biomarker between cAbs at the electrode surface and dABs. By way of example, FIG. 6C illustrates a detection operation 650 via which SWV is used to measure the amount of TH bound to the working electrode surface. As depicted in this example, as TH molecules are directly conjugated to CRP dAb-immobilized AuNPs, their amount bound is directly correlated to the amount of CRP ‘sandwiched’ between cAbs at the electrode surface and dAb-immobilized AuNPs, and consequently, the initial concentration of CRP in solution.

Depending on the binding environment, there may be significant interpersonal variations in the composition of the biofluid sample, which could affect the rate that biomarkers bind to detection reagents, and affect the accuracy of the estimated concentration of the biomarker. For example, as further discussed below, it was found during experimentation that pH, electrolyte concentration, and temperature can all influence the sensor readout of CRP concentration expressed as a current measurement. As such, in some implementations, to further improve the quantification of biomarkers contained in the biofluid sample, the influence of temperature, pH, and/or ionic strength on the biomarker sensor readings can be calibrated in real-time based on readings from temperature sensor 122, pH sensor 123, and/or ionic strength sensor 124 of the biofluid sample in detection reservoir 134.

In some implementations, to mitigate the difference in binding environment, electrolytes can be introduced into the detection reservoir 134. For example, high-level buffering salts can be deposited with dAbs in a reagent reservoir to mitigate potential binding environment changes caused by sweat composition variations.

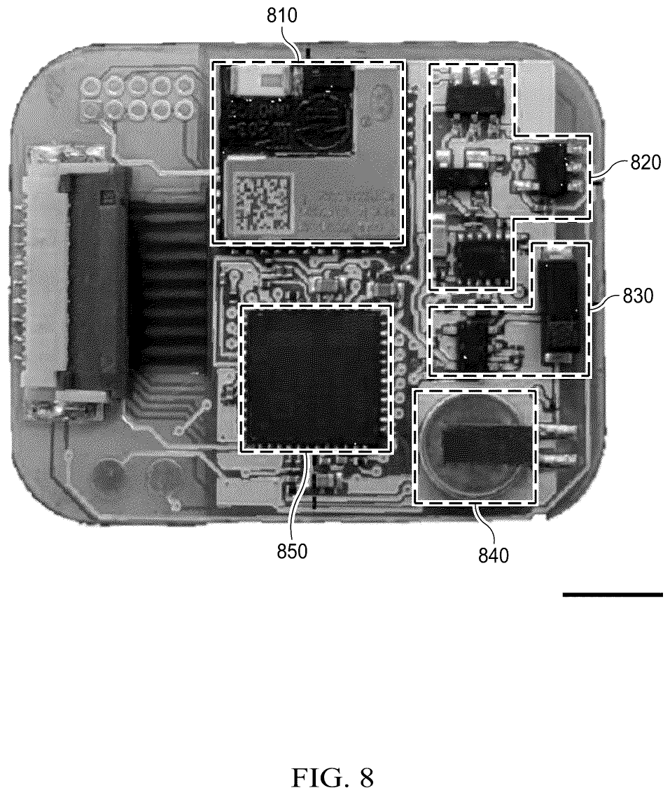

FIG. 8 illustrates an enlarged plan view of the electronics that can be implemented in a FPCB 200, in accordance with a particular embodiment. As depicted by the dashed lines indicating different modules, the FPCB 200 can include a signal processing and wireless communication module 810, an iontophoresis module 820, a power management module 830, a battery 840, and an electrochemical sensor instrumentation module 850. In this example, the scale bar is 5 mm.

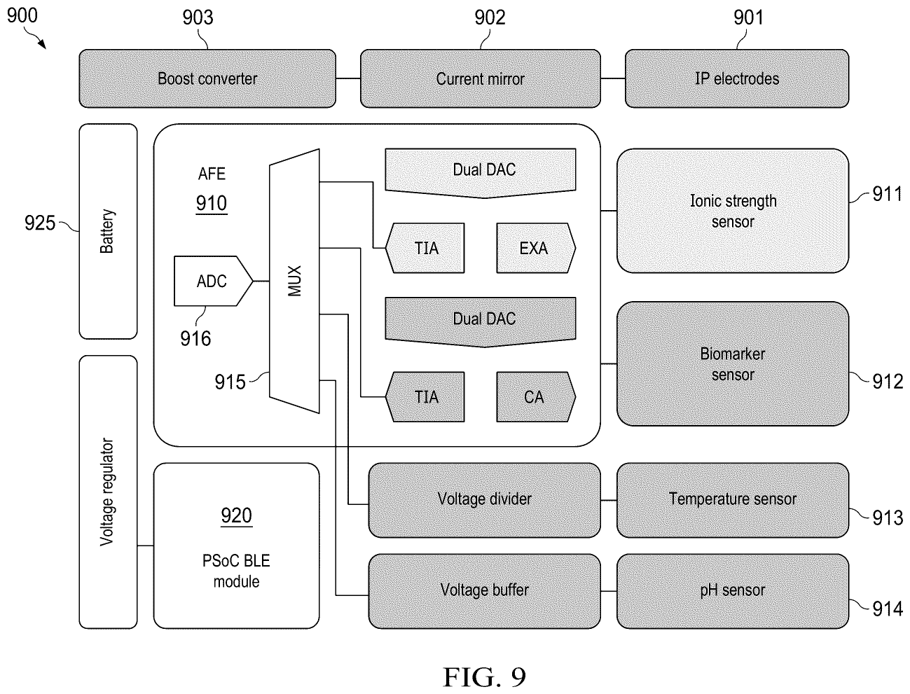

FIG. 9 is a block diagram illustrating an example electronic system 900 of a biosensor device 300 used for CRP sensing, in accordance with a particular embodiment. As depicted, the components of electronic system 900 can be powered using battery 925. The electronic system 900 includes iontophoresis (IP) electrodes 901 for iontophoresis sweat collection. The IP electrodes 901 can be electrically coupled to a current mirror 902 and boost converter 903. The electronic system also includes a multiplexed sensor array including an ionic strength sensor 911, biomarker sensor 912, temperature sensor 913, and pH sensor 914, that generate signals routed to multiplexer 915, e.g., after signal processing. In this example, the multiple sensors are interfaced to analog-to-digital converter 916 using an analog-front-end 910. In this example, wireless communication is implemented using a programmable system on a chip (PSoC) BLE module 920. Via the illustrated electronics, a FPCB can be configured to perform current-controlled iontophoresis, multiplexed electrochemical measurements (including voltammetry, impedimetry, and potentiometry), signal processing, and wireless communication. The system could also accurately obtain the dynamic responses of integrated LEG-based pH, ionic strength, and skin temperature sensors for real-time CRP sensor calibration.

FIG. 10 illustrates an example graphical user interface (GUI) 1000 that can be presented to a user (e.g., patient) by running a mobile application used in conjunction with a wearable biosensor device 300 for noninvasive automatic biomarker monitoring, in accordance with some implementations of the disclosure. For example, the application can be run by a mobile device 50 wirelessly coupled to a biosensor device 300. During runtime, the GUI can display real-time data (processed or otherwise) acquired by the biosensor device 300. The GUI can also display historical data that was acquired. For example, based on a sweat sample collected by the biosensor device 300, data such as a CRP concentration (e.g., in ng/mL), a pH, and a skin temperature can be acquired and presented in real-time. The data can be plotted over time to provide an indication of the user's inflammation levels or other biological levels over time. The GUI can provide an indication of whether the user's measured health data is within a normal or abnormal range (e.g., via textual or visual markers). The GUI can also provide an indication of the status of the biosensor device 300 (e.g., whether it is presently connected to the mobile device).

In some implementations, the mobile application can itself perform, prior to user display, processing of sensor measurements received from a biosensor device 300. For example, in one implementation, the mobile application can be configured to convert a biomarker concentration based on an obtained voltammogram (e.g., SWV voltammogram) and corresponding real-time obtained values of calibration sensors such as an ionic strength sensor, pH sensor, and temperature sensor.

In some implementations, sweat samples can be collected without reapplication of a hydrogel agent for a period of time. A period of time may be from about two hours up to a full, twenty-four hour day. Refreshed samples can be periodically or continuously collected in the microfluidic patch, mixed with labeled reagents, channeled into a detection reservoir, analyzed, and then flushed out through an outlet. The entire process illustrated above can be merged and integrated on a single sweat sensor patch. After a full day or other time period, a new sweat sensor patch with a new hydrogel agent may be applied and the foregoing process for biomarker detection repeated. The process can be repeated on a daily basis for an extended period of several days, weeks, or even months. The process can also be resumed after a break of a period of days, weeks, or months, to evaluate a change in a medical condition.

In some implementations, a microfluidic sweat collection patch may be optimized to achieve the most rapid refreshing time between samples. Several parameters may be selected for optimization. These parameters may include, for example, the placement of inlet(s) relative to each other and a reagent reservoir, the shape and distance of the mixing channel, a number of inlet(s), the distance between an inlet and reagent reservoir, the shape and distance of the mixing channel, the shape and size of the detection reservoir, the placement and distance of an outlet relative to a detection reservoir, and other factors.

In some implementations, a microfluidic sweat collection patch may be designed to eliminate leakage of a sweat sample. For example, the electrostimulation may be applied to several neighboring sweat glands while avoiding the sweat glands directly underneath inlets. The patch may be designed to allow for collection of a sweat sample from only glands not in touch with the hydrogels and prevent leakage of sweat from the neighboring sweat glands (which mixed with hydrogel). This may be achieved through application of pressure on the gland the sample is taken from and through application of specialized adhesive taping of the neighboring glands and use of secure adhesive to attach the skin patch. The application of hydrogel may also be limited to optimal parts of the patch to minimize interference.

In some implementations, when necessary, dynamic and automatic wearable biomarker sensing could be realized by incorporating capillary bursting valves and sensor arrays into a single disposable sensor patch.

### EXPERIMENTAL AND SIMULATION RESULTS

Various experiments and simulations were performed using a biosensor device 300 and/or components thereof used to wirelessly, autonomously, and non-invasively monitor CRP levels, in accordance with a particular embodiment of the disclosure. The design of this particular biosensor device 300, and its associated experimental and simulations results, are further detailed below. Although these experimental and simulation results exemplify some of the advantages of utilizing the technology described herein, it should be appreciated that the disclosure is not limited by the discussion that follows, which describes results and observations of utilizing particular example embodiments. For example, besides CRP, this wearable approach could be adapted to assess other trace-level disease-relevant protein biomarkers on-demand. Additionally, the operation principle described herein could be readily adapted to survey a broad array of biomarkers (e.g., proteins, hormones, cytokines, etc.), including biomarkers that indicate the presence of inflammation or some other biological condition.

Fabrication of a Multiplex Microfluidic Sensor Patch

A particular embodiment of a microfluidic sensor patch was fabricated as follows. A PI film was raster engraved at focus height (8% Power, 15% Speed, 1000 Points Per Inch) to fabricate LEG-based iontophoresis IP electrodes, connection leads, impedance, CRP working, counter and reference electrodes using a 50 W CO₂ laser cutter. pH electrode and temperature sensors were engraved using vector mode with 1% and 3% Power, respectively (15% Speed, 1000 Points Per Inch (PPI)). The working electrode of the pH sensor was prepared by electrochemically cleaning the LEG electrode in 1M HCl via cyclic voltammetry from −0.2 to 1.2 V at 0.1V s⁻¹ for 10 cycles followed by electrodeposition of polyaniline pH sensing membrane via cyclic voltammetry from −0.2 to 1.2 V at 0.1 V s⁻¹ for 10 cycles. A shared Ag/AgCl reference electrode was fabricated by electrodeposition of Ag on the LEG electrode in a solution containing silver nitrate, sodium thiosulfate, and sodium bisulfite (250 mM, 750 mM, and 500 mM, respectively) using multi-current steps (30 s at −1 μA, 30 s at −5 μA, 30 s at −10 μA, 30 s at −50 μA, 30 s at −0.1 mA and 30 s at −0.2 mA), followed by drop casting 10 μL-aliquot of 0.1M iron chloride (III) for 1 minute. AuNPs were electrodeposited on the LEG CRP working electrode via pulse deposition (two 0.5 s pulses at −0.2 V separated by a 0.5 s pulse at 0 V) for 40 cycles in the presence of 0.1 mM gold(III) chloride trihydrate and 10 mM sulfuric acid.

Iontophoresis hydrogels containing cholinergic agent carbachol (placed on the IP electrodes) were prepared by dissolving agarose (3% w/w) in deionized water using a microwave oven. After the agarose was fully dissolved, the mixture was cooled down to 165° C. and 1% carbachol for anode (or 1% KCl for cathode) was added to the above mixture and stirred to homogeneity. The cooled mixture was casted into cylindrical molds or assembled microfluidic patch and solidified at room temperature. The hydrogels were stored at 4° C. until use.

A microfluidic module was prepared with an assembly of thin PET film (50 μm) sandwiched between double-sided medical adhesives (180 μm top layer, 260 μm bottom layer with a 50 μm PET backing) that was attached to a substrate and cut through to make channels and reagent reservoirs using a laser cutter at 2.7% power, 1.8% speed, 1000 PPI vector mode. Next, 4% power, 10% speed, 1000 PPI vector mode was used to cut a circular outline through only the top layer of medical adhesive (180 μm). The circular top layer was peeled off to make the detection reservoir. A sweat accumulation layer was prepared by cutting through a 130 μm adhesive. Labeled dAb-AuNPs were drop-casted and dried in the reagent reservoir and stored in dry state at 4° C. before assembly with the sensor patch.

LEG-AuNPs CRP Working Electrode Functionalization

In one particular embodiment, LEG-AuNPs CRP working electrodes were functionalized as follows. LEG-AuNPs working electrodes were immersed in 0.5 mM mercaptoundecanoic acid (MUA) and 1 mM mercaptohexanol (MCH) in proof 200 ethanol overnight for SAM formation. After rinsing with ethanol followed by deionized (DI) water and drying under airflow, electrodes were incubated with 10 μL of a mixture solution containing 0.4 M N-(3-Dimethylaminopropyl)-N′-ethylcarbodiimide (EDC) and 0.1 M N-hydroxysulfosuccinimide sodium salt (sulfo-NHS) in 25 mM 2-(N-morpholino)ethanesulfonic acid hydrate (MES) buffer, pH 5.0, for 35 minutes at room temperature in a humid chamber. Covalent attachment of CRP cAbs was carried out by drop casting 10 μL of anti-CRP solution (250 μg mL⁻¹ in phosphate-buffered saline (PBS), pH 7.4) and incubated at room temperature for 2.5 hours, followed by a 1-hour blocking step with 1.0% bovine serum albumin (BSA) prepared in PBS. Electrodes were stored in 1% BSA in PBS until use.

CRP Detector Antibody Conjugation

In one particular embodiment, CRP detector antibody conjugation was achieved as follows. 20 nm carboxylic acid functionalized PEGylated gold AuNPs were activated with EDC/Sulfo-NHS mix solution (30 mg mL −1 and 36 mg mL⁻¹ respectively) in 10 mM MES buffer (pH 5.5) for 30 minutes. The conjugates were washed with 1×PBS containing 0.1% Tween® 20 (PBST) and centrifuged at 6500 relative centrifugal force (rcf) for 30 minutes. After supernatant removal, 50 μg mL⁻¹ polystreptavidin R (PS-R) was added and allowed to crosslink for 1 hour at room temperature. Following centrifugation at 3500 rcf for 30 minutes and supernatant removal, 5 μg mL⁻¹ biotinylated anti-CRP dAb in 1% BSA prepared in 1×PBS (pH 7.4) was incubated for 1 hour at room temperature. After another round of washing (centrifugation at 2000 rcf), the carboxyl groups of PS-R and dAb on AuNP were activated with EDC/Sulfo-NHS mix solution (30 mg mL −1 and 36 mg mL⁻¹ respectively) in 10 mM MES buffer (pH 5.5) for 30 minutes. After the washing step using centrifugation at 1500 rcf, 100 μM thionine was incubated for 1 hour. The final conjugate was washed with PBST, centrifuged at 1250 rcf, reconstituted in 1% BSA and filtered through 0.2 μm syringe filter to remove all aggregates.

For direct redox probe conjugation to antibodies, 100 μg mL⁻¹ dAb was buffer exchanged by concentrating with a 100K MWCO protein concentrator and reconstituted in 10 mM MES buffer (pH 5.5). The carboxyl groups of dAb were activated with EDC/Sulfo-NHS mix solution (30 mg mL⁻¹ and 36 mg mL⁻¹ respectively) in 10 mM MES buffer (pH 5.5) for 30 minutes in column. Following buffer exchange with 1×PBS (pH 7.4), 100 μM thionine was incubated for 1 hour. The final conjugate was buffer exchanged with PBS, reconstituted in 1% BSA, and filtered through 0.2 μm syringe filter to remove all aggregates.

Electronic System Design and Integration

In one particular embodiment, the electronic system was designed as follows. A 2-layer flexible printed circuit board FPCB was designed. The FPCB outline was designed as a rounded rectangle (31.7 mm×25.5 mm), the same size as the microfluidic sensor patch such that the patch can be inserted directly underneath the FPCB via a cutout (10 mm×3.8 mm). The electronic system was composed of a magnetic reed and a voltage regulator for power management; a boost converter, BJT array, and analog switch for iontophoretic induction; an electrochemical front-end, an operational amplifier, and a voltage divider for sensor array interface; and a BLE module for system control and Bluetooth wireless communication. A BLE connection was established with the wearable device and to wirelessly acquire sensor data for calibration and voltammogram analysis. A rechargeable 3.8 V lithium button cell battery with capacity of 8 mAh was used to power the electronic system. To reduce the existing noise caused by motion artifacts, filtering and smoothing techniques were employed. On the hardware side, the electrochemical AFE filtered noise from the ADC via digital filters. On the software side, smoothing algorithms (moving average filter/median filter) were automatically applied in real-time.

Electrochemical Characterizations of LEG-AuNPs Immunosensor

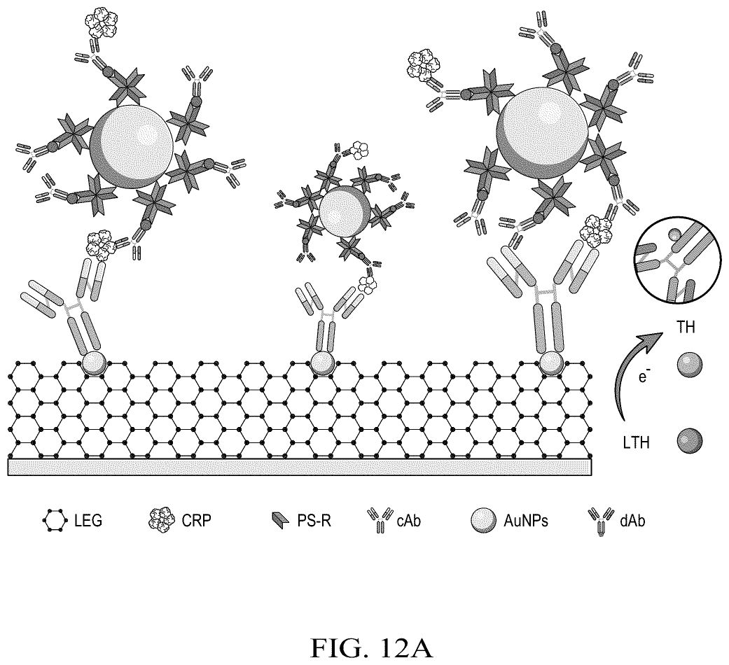

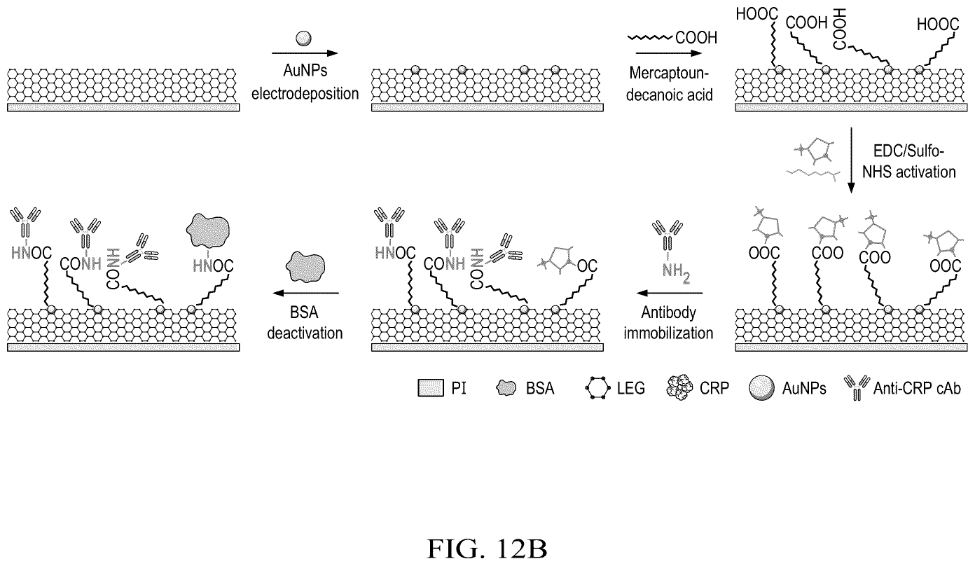

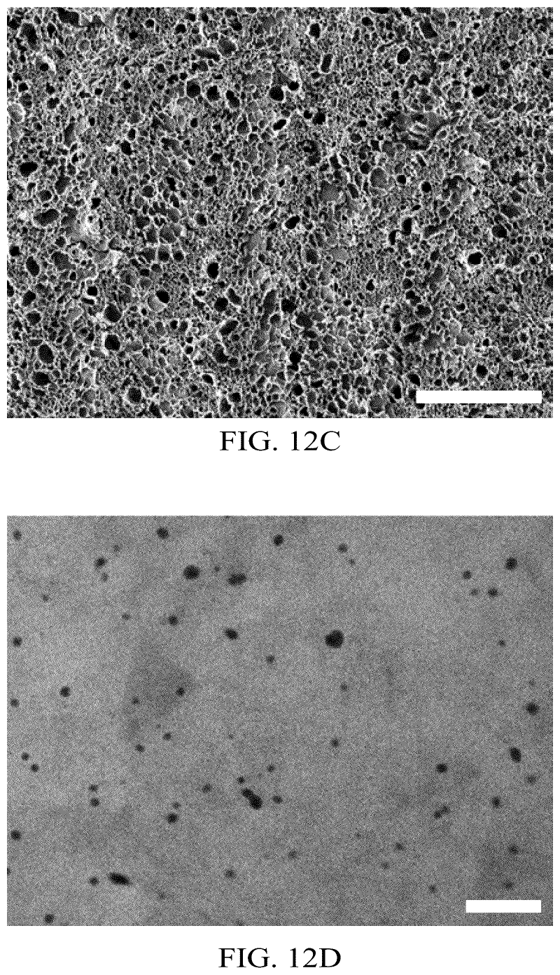

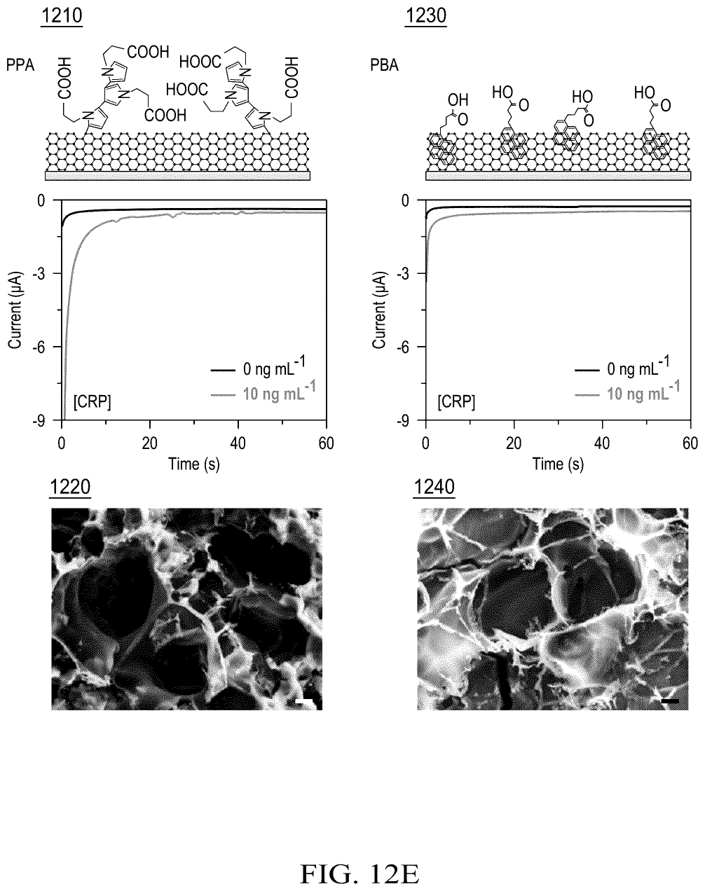

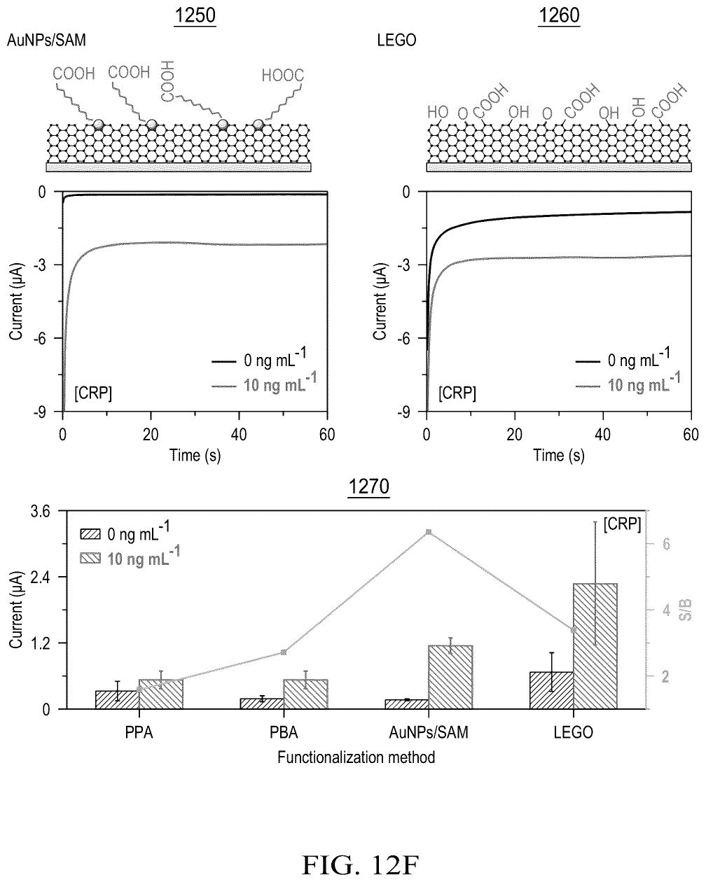

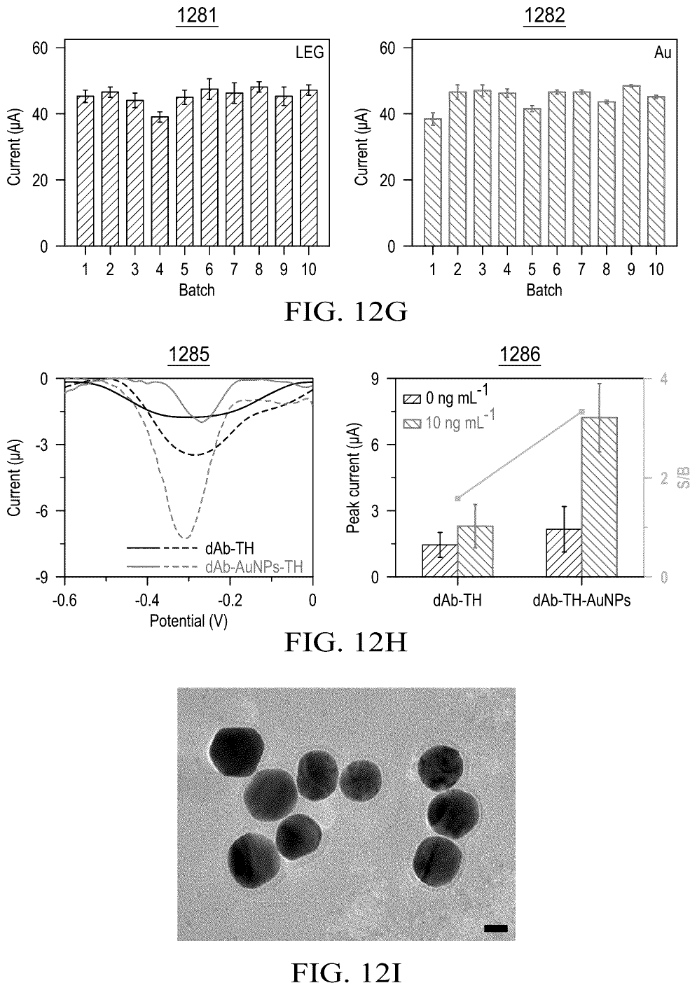

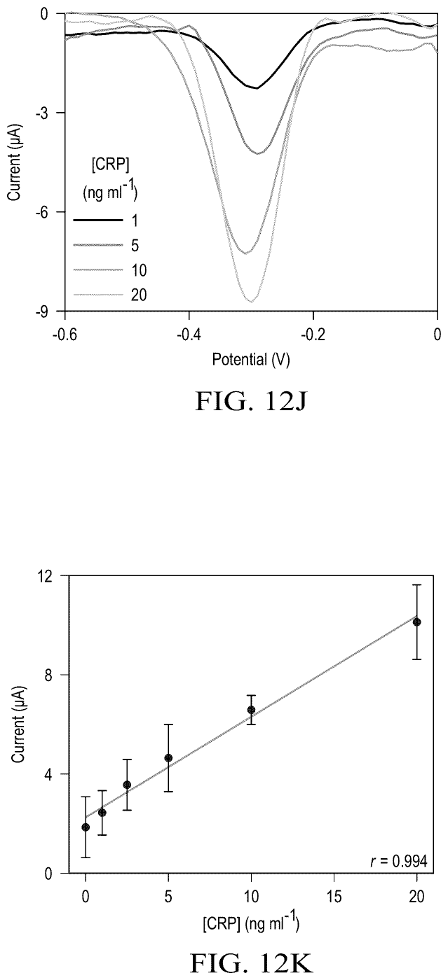

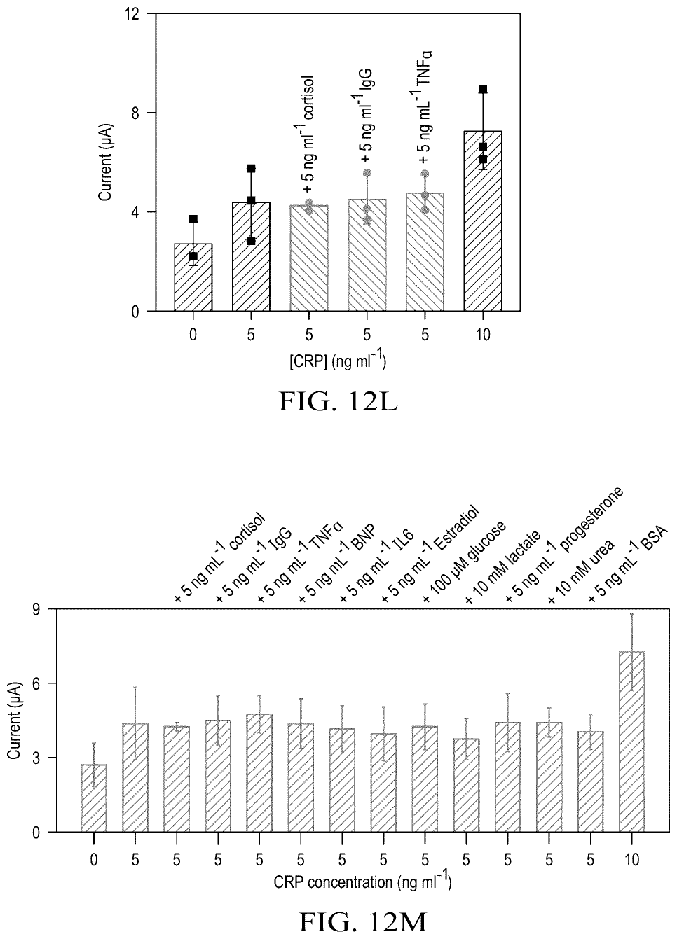

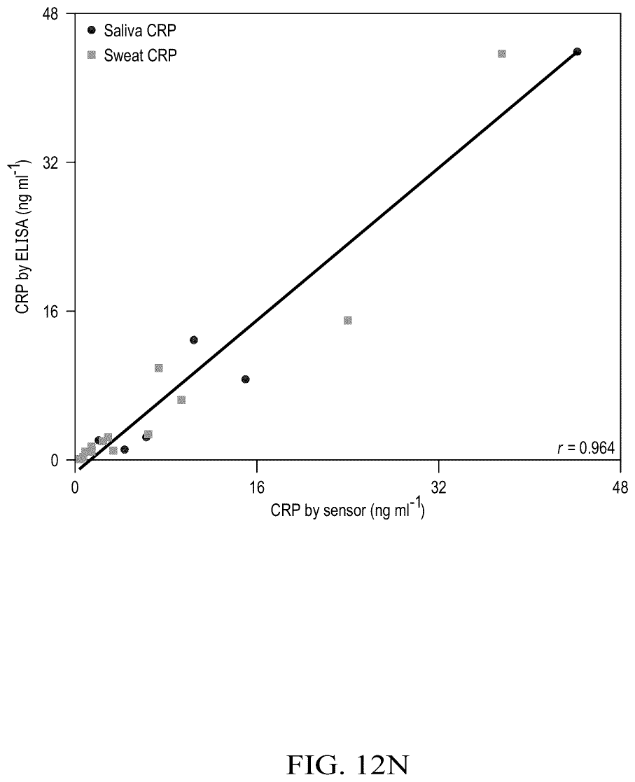

FIGS. 12A-12B illustrate the surface functionalization process of the LEG-AuNPs working electrode of a CRP sensor, in accordance with a particular embodiment. As depicted, the AuNPs can be electrodeposited on the LEG surface followed by subsequent thiol monolayer assembly with mercaptoundecanoic acid and mercaptohexanol. As the formation of the SAM layer can rely on specific gold-sulfur bonding, it was observed that immersion of the sensor patch in alkanethiol solution had negligible influence on other graphene-based electrodes. As illustrated by FIG. 12C (showing SEM image of mesoporous LEG electrode, with scale bar of 100 μm), FIG. 12D (showing transmission electron microscopy image of AuNP-decorated graphene flakes, with a scale bar of 50 nm) and image 1120 of FIG. 11, it was also observed that pulsed potential-deposited AuNPs evenly distributed throughout the mesoporous graphene structure and possessed superior electrocatalysis capability and formed a large number of binding sites on the surface of the particles for biomolecule immobilization.

As depicted by FIGS. 12E-12F, it was also observed that an LEG CRP sensor prepared by a functionalization method that relied on the LEG-AuNPs composited modified with the thiol SAM (AuNPs/SAM) achieved superior electrochemical performance relative to other functionalization methods, substantially improving sensitivity of the CRP sensor with little non-specific adsorption. FIG. 12E illustrates amperometric responses and SEM images of CRP sensors based on the LEG modified with PPA (1210, 1220), and PBA (1230, 1240). FIG. 12F illustrates amperometric responses of CRP sensors based on AuNPs/SAM (1250), and laser-engraved graphene oxide by electrochemical oxidation (LEGO) (1260). FIG. 12F also includes a plot 1270 illustrating a sensor performance comparison of the different functionalization methods, where error bars represent the s.d. of the mean from 3 sensors, and S/B is the signal to background ratio.

The formation of LEG-AuNPs composite was observed through the increased ratio of the intensity of D and G bands in the Raman spectra due to the presence of AuNPs. The individual sensor modification steps on the LEG electrodes were characterized with X-ray photoelectron spectroscopy. It was observed that the intensity of Au4f increases substantially after the deposition of AuNPs while N1s increases only after the cAb immobilization step, indicating successful electrode preparation. DPV and electrochemical impedance spectroscopy (EIS) were used to further characterize the LEG surface electrochemically after each modification step. It was observed that there was a decrease in peak current height in DPV voltammograms and increased resistance in Nyquist plots after SAM and cAb protein immobilization, indicating that SAM and cAb impeded the electron transfer at the interface. This was due to the increase in surface coverage by non-conductive species. Moreover, it was found that negatively charged carboxylate functional groups in the SAM layered result in the repulsion of the negatively charged redox indicator, ferricyanide, and further reducing the electron transfer rate. Subsequent modification of the SAM layer with EDC/NHS chemistry replaces the negatively charged carboxylate groups with neutral NHS-ester groups. This was empirically observed as an increase in peak current height. As depicted by FIG. 12G, which shows, batch to batch variations in electrochemical performance of the LEG electrodes and LEG-AuNPs electrodes, such electrode fabrication processes showed high batch-to-batch reproducibility as the main processes including laser engraving, electrochemical deposition, and solution process were all mass-producible. In particular, FIG. 12G, includes plots showing oxidation peak heights in the CVs of the LEG electrodes (plot 1281) and LEG-AuNPs electrodes (plot 1282), 0.1 M KCl and 5 mM [Fe(CN)₆]³⁻, Scan rate, 50 mV s⁻¹, where bars represent the s.d. of the mean from 3 sensors.

In this particular embodiment, to realize trace-level sweat CRP analysis, PEGylated AuNPs that possess large surface area-to-volume ratio were functionalized with PS-R to increase the loading of biotinylated-dAbs and subsequently enhance sensitivity. For example, FIG. 12H includes plots 1285-1286 showing a comparison of the electrochemical performances of redox probe conjugated dAb and dAb-conjugated AuNPs. Plot 1285 shows SWV voltammograms of the CRP sensors modified with redox probe conjugated dAb and dAb-conjugated AuNPs. Plot 1286 shows corresponding peak currents of the CRP sensors modified with redox probe conjugated dAb and dAb-conjugated AuNPs. In the plots, solid lines and dotted lines represent the sensor responses in 0 and 10 ng mL⁻¹ CRP, respectively. Error bars represent the s.d. of the mean from 3 sensors.

In this particular embodiment, one-step direct electrochemical detection was enabled by crosslinking the redox label TH onto the carboxylate residues on the dAb-loaded AuNPs. As the TH-labeled dAb-loaded AuNPs bound to the mesoporous graphene electrode upon CRP recognition, TH located on the external sites of the proteins were in close proximity to the graphene surface in each mesopores for electron transfer. The successful immobilization of the dAbs was confirmed based on a variety of observations. For example, the successful immobilization was confirmed from observed increases in hydrodynamic sizes of the PEGylated AuNPs after each conjugation step by dynamic light scattering: PS-R immobilization, biotinylated dAb binding and redox molecule TH conjugation followed by BSA deactivation. The successful immobilization of the dAbs was also confirmed from observed shifts of ultraviolet-visible (UV-Vis) absorbance of the AuNPs conjugate after each modification step, and from a TEM image showing dispersed dAb-loaded AuNPs with protein corona shells (FIG. 12I).

The performance of the CRP in this particular embodiment was evaluated with SWV in CRP spiked PBS solutions (FIG. 12J), and increases in peak current height of TH reduction were observed to show a linear relationship with increased target concentrations (FIG. 12K). In particular, FIG. 12J shows SWV voltammograms and FIG. 12K shows the corresponding calibration plot of the CRP sensors in 1×PBS (pH 7.4) with 0-20 ng ml⁻¹ CRP and 1% BSA, where error bars represent the s.d. of the mean from three sensors. In this particular embodiment, it was observed that the sensor could detect picomolar levels of CRP with an ultralow limit detection on the order of about 8 pM. When performing detection in 10 batches of 1×PBS (pH 7.4) in the presence of 0 and 5 ng mL⁻¹ CRP, it was also observed that the sensor demonstrated good batch-batch reproducibility. It is anticipated that sensing accuracy of this particular embodiment could be further enhanced by automating the sensor preparation and modification process (e.g., via automated fluid dispensing or inkjet printing).

It was also observed that the LEG-AuNPs CRP immunosensor demonstrated high selectivity over other potential interference proteins and hormones attributed to the sandwich assay format. For example, FIGS. 12L and 12M are plots illustrating the selectivity of the CRP sensor to potential interferences in sweat, where the errors bars represent the s.d of the mean from three sensors. Considering interpersonal variations during the human study, the influence of sweat pH, ionic strength, temperature, and sample volume on the antibody-antigen binding kinetics and redox probe electron transfer rate on CRP sensing accuracy was investigated and mitigated by introducing suitable calibration mechanisms, further described below. The potential variations of using a Ag/AgCl pseudo-reference electrode in the presence of varying Cl⁻ concentration in the physiologically-relevant range were found to result in a small shift in the peak potential but its influence on the overall peak current density (and thus CRP quantification) was found to be negligible. As depicted by FIG. 12N, which is a plot showing validation of the CRP sensor in human sweat samples (n=13 biological replicates) and saliva samples (n=6 biological replicates), the accuracy of the CRP sensor for biofluid analysis was validated by the laboratory gold standard enzyme-linked immunosorbent assay (ELISA) using the human sweat and saliva samples. It was also observed that the disposable CRP sensors maintained stable sensor performance over a 10-day period when stored in PBS in a refrigerator at 4° C.

Evaluation of Sweat CRP for Non-Invasive Monitoring of Systemic Inflammation

Inflammatory processes and immune responses are associated with a broad spectrum of physical and mental disorders that contribute substantially to modern morbidity and mortality globally. The top three leading causes of death worldwide, namely, ischemic heart disease, stroke, and COPD, are each characterized by chronic inflammation. Although the acute inflammatory response is a critical survival mechanism, chronic inflammation contributes to long-term silent progression of disease through irreversible tissue damage. Delayed diagnosis and treatment of chronic diseases impose heavy financial burdens on patients and the healthcare systems.

Although there is no canonical standard biomarker for the measurement and prediction of systemic chronic inflammation, CRP, an acute-phase protein synthesized by hepatocytes in response to a wide range of both acute and chronic stimuli, has a close association with chronic inflammation and respective risks of mortality in several disease states. The stable nature of CRP in plasma, the absence of circadian variation, and its insensitivity to common medications such as corticosteroids render it extremely attractive to clinicians as a handy means to assess a patient's physiological inflammatory state. There is also a growing interest in exploring the effectiveness of serial CRP measurements for therapeutic decision-making.

At present, circulating CRP levels are clinically assessed in specific laboratories that rely on invasive blood draws from patients. Commercial point-of-care CRP monitors are still bulky in size and cannot reach picomolar-level sensitivity to assess CRP levels in non-invasively accessible alternative biofluids such as sweat and saliva. A readily available means of monitoring inflammatory biomarkers such as CRP at home could improve patient outcomes and lower cost factors by monitoring disease progression and initiating early treatment and intervention.

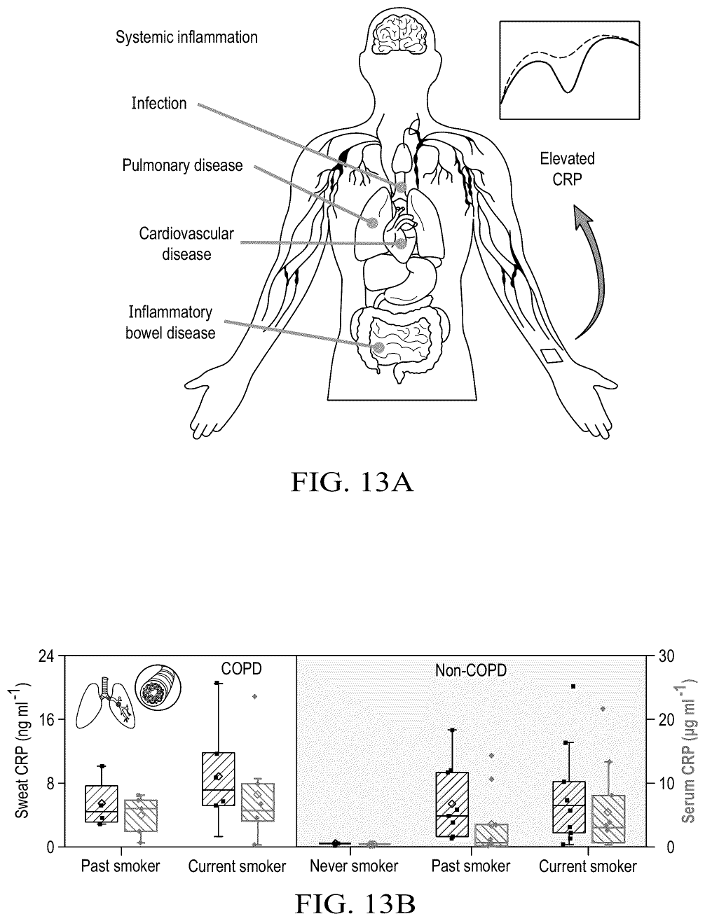

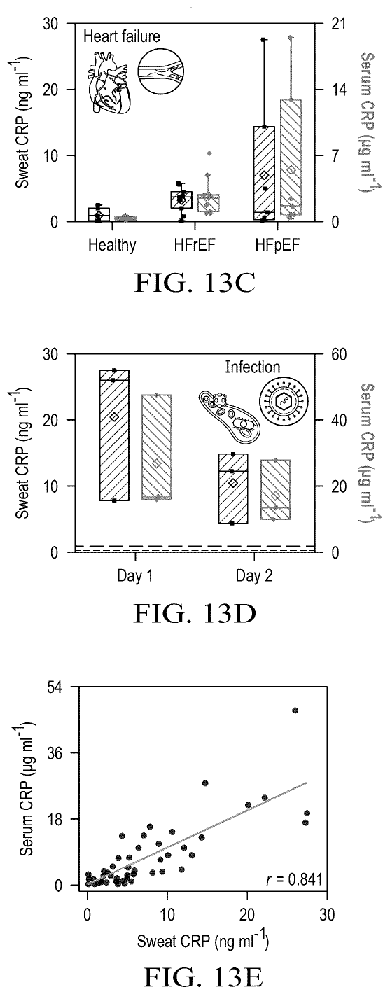

As such, the use of LEG-AuNPs CRP sensors for the assessment of sweat CRP as a universal, cost-effective, and non-invasive approach to monitor systemic inflammation in various disease states was evaluated. For example, FIG. 13A depicts a high level schematic of the evaluation of sweat CRP for the non-invasive monitoring of various health conditions that could be associated with elevated CRP in healthy or patient populations, including infection, pulmonary disease, cardiovascular disease, and inflammatory bowel disease.

Prior to performing these evaluations, a proteomic characterization of different types of sweat samples using bottom-up proteomic analysis was conducted to affirm the presence of CRP in sweat generated by iontophoresis and by vigorous exercise. Using a recombinant CRP protein standard as the reference, CRP was identified in both exercise and iontophoretic sweat samples from human subjects.

In one study, using the LEG AuNPs CRP sensor, CRP levels were evaluated in healthy subjects grouped according to smoking status (current, former, and never smokers). Results of the study are illustrated by FIG. 13B, which includes a box-and-whisker plot of CRP levels in iontophoresis-extracted sweat and serum samples from patients with COPD (n=10 biological replicates) and without COPD (n=24 biological replicates). The participants were classified into five subgroups: current smokers with COPD (n=6 biological replicates) or without COPD (n=10 biological replicates), former smokers with COPD (n=4 biological replicates) and without COPD (n=9 biological replicates) and never smokers without COPD (n=5 biological replicates). It was observed that CRP levels in both serum and sweat were greater in current smokers as compared with former and never smokers, consistent with previous reports on the effect of current smoking on serum CRP. Among COPD patients, it was observed that serum and sweat CRP values were greater in former smokers than current smokers, consistent with irreversible tissue damage and chronic inflammation in COPD patients even after smoking cessation. The foregoing experimental results illustrate that monitoring sweat CRP in COPD patients could therefore be useful for following disease progression and/or predicting exacerbation in this patient population.

In another, preliminary study, using the LEG AuNPs CRP sensor, CRP levels were evaluated in heart failure (HF) patients. Chronic systemic inflammation can be related to increased risks of cardiovascular events. Results of the study are illustrated by FIG. 13C, which includes a box-and-whisker plot of CRP levels in sweat and serum samples from healthy participants (n=7 biological replicates), patients with HF with reduced ejection fraction (HFrEF; n=7 biological replicates) and patients with HF with preserved ejection fraction (HFpEF; n=9 biological replicates). The sensor results showed that that serum and sweat CRP values were substantially elevated in patients with HFpEF but not in patients with HFrEF, consistent with past studies. The foregoing experimental results illustrate that the investigation of the dynamics of sweat CRP using the technology described herein could potentially have high value in predicting HFpEF disease progression and clinical outcomes.

In addition to chronic infections in COPD and HF, acute infections (such as COVID-19) could lead to severe inflammatory responses. In a further, pilot study, using the LEG AuNPs CRP sensor, CRP levels were evaluated in hospitalized patients with active infections for two consecutive days. Results of the study are illustrated by FIG. 13D, which includes a box-and-whisker plot of CRP levels in sweat and serum samples from three patients with active infection on two consequent days (n=3 biological replicates). The dotted lines represent the mean values of the sweat and serum CRP levels for healthy participants. Substantial increase (over 10-fold on average) in both serum and sweat CRPs was identified in patients with active infection as compared with healthy subjects, indicating the presence of highly elevated sweat CRP in acute inflammation. In the plots of FIGS. 13B-13D, the bottom whisker represents the minimum, the top whisker represents the maximum and the square in the box represents the mean.

In a further study, using the LEG AuNPs CRP sensor, CRP levels were analyzed in samples from healthy subjects and patient populations with various inflammatory conditions. Results of the study are illustrated by FIG. 13E, which is a plot showing correlation of serum and sweat CRP levels, where the correlation coefficient was acquired through Pearson's correlation analysis (n=80, P<0.00001). Using the CRP sensor, a high correlation coefficient (r) of 0.844 between sweat and serum CRP concentrations was obtained. Such correlation to serum CRP concentrations appeared to be higher than those obtained from saliva and urine samples in one study, suggesting the great potential of using sweat CRP for the non-invasive monitoring of systemic inflammation toward the management of a variety of chronic and acute health conditions.

Clinical On-Body Evaluation

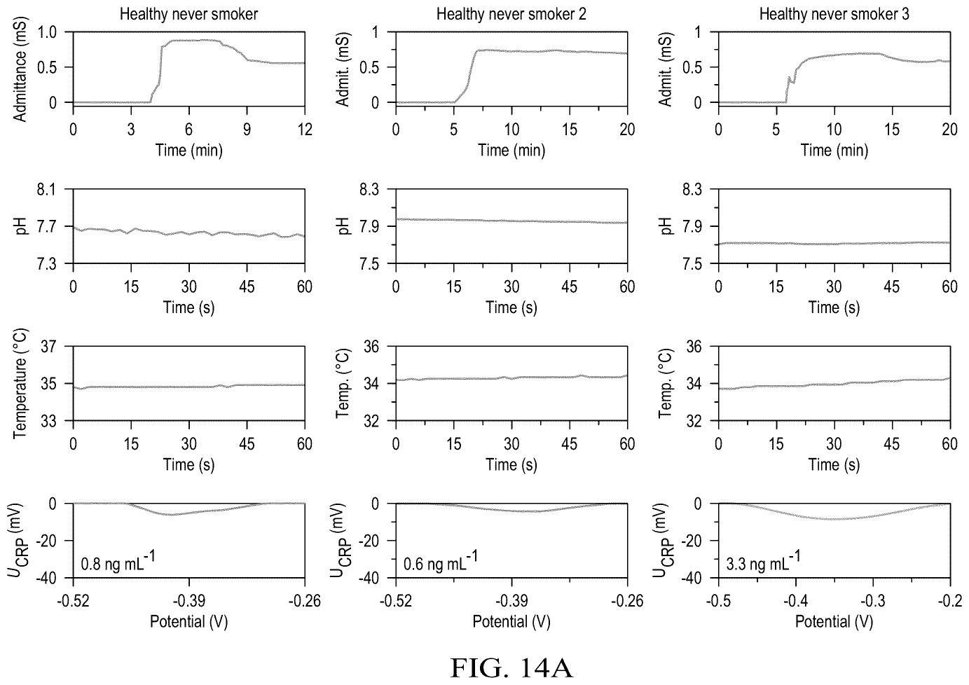

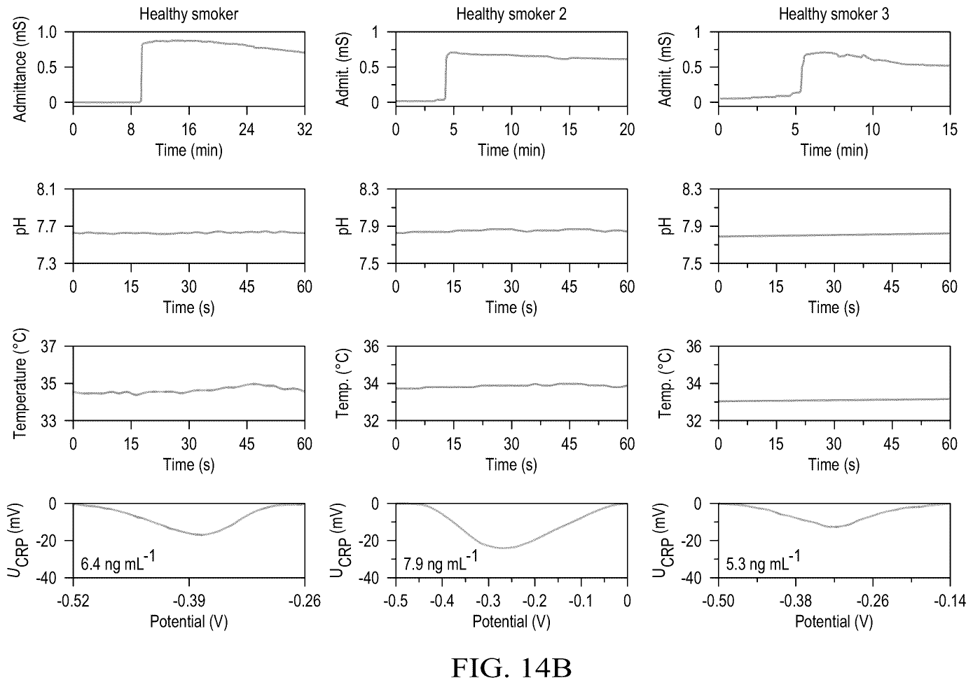

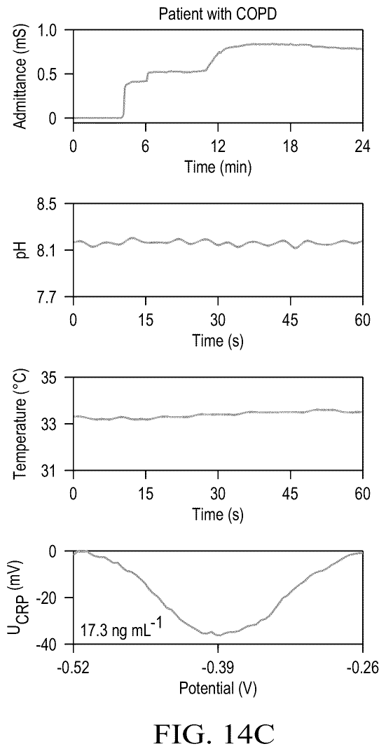

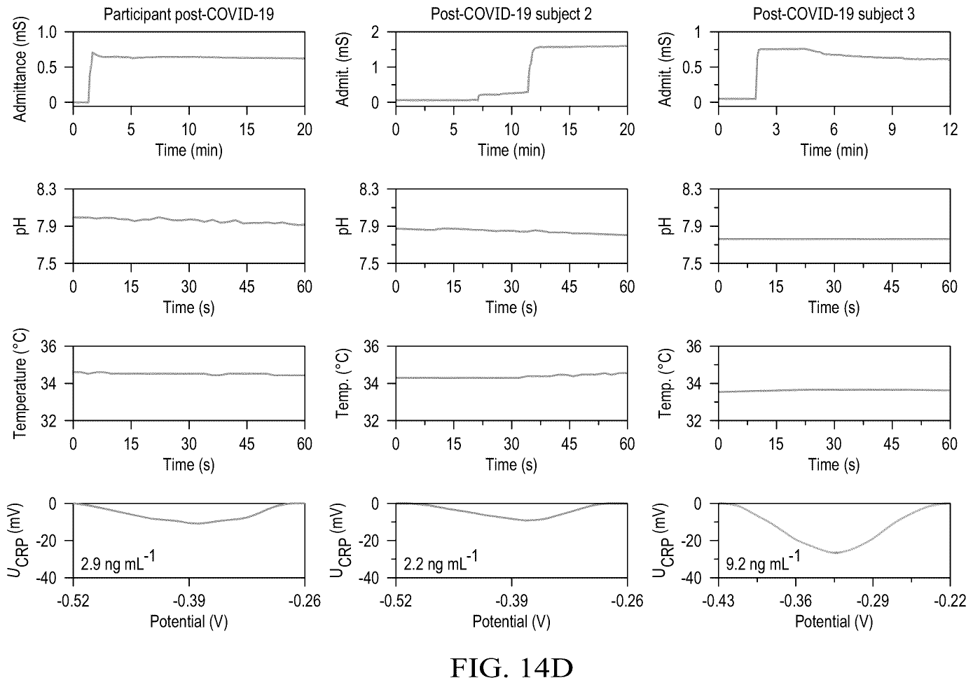

Clinical on-body evaluation of a wearable biosensor system including a multiplexed LEG sensor array was performed on healthy subjects (involving both never smokers and current smokers) as well as patients with COPD and post-COVID-19 infection. Some of the results of on-body evaluation of the multiplexed sensor patch toward noninvasive automatic inflammation monitoring are illustrated in FIGS. 14A-14D, which show on-body multiplexed physicochemical analysis and CRP analysis with real-time sensor calibrations using the wearable sensor from healthy never smokers (FIG. 14A), healthy smokers (FIG. 14B), a patient with COPD (FIG. 14C), and participants who previously had COVID-19 (FIG. 14D). During on-body trials, it was observed that the wearable system laminated conformally on the subject's arm, chemically inducing and analyzing sweat, and acquiring inflammatory biomarker information non-invasively and wirelessly. In the foregoing trials, in situ pH, temperature, and CRP sensor readings were acquired after the ionic strength sensor indicated full refreshment of the detection reservoir. The CRP concentration was converted in a mobile application based on the obtained SWV voltammogram and the corresponding real-time obtained ionic strength, pH, and temperature values. As expected, an elevated CRP level was observed from the current smokers as compared with the never smokers in healthy subjects. The CRP levels in the COPD patients and post-COVID subjects were substantially greater than those of non-smoking healthy subjects, suggesting the promise of using the biosensor device 300 in practical non-invasive systemic inflammation monitoring and disease management applications. In vitro analysis of sweat and serum from post-COVID subjects corroborated the on-body observation that patients who experienced moderate symptoms during COVID may still present a low-grade inflammation post COVID episode as indicated by the slightly elevated CRP levels. Similar as serum, it was observed that sweat CRP levels remained substantially stable during a 30-minute test period and no substantial variations were observed for chemically-induced sweat samples at different body locations, including the forearm, leg, upper arm location, thigh, and back.

Characterization of Multiplexed Microfluidic Patch for Automatic Immunosensing

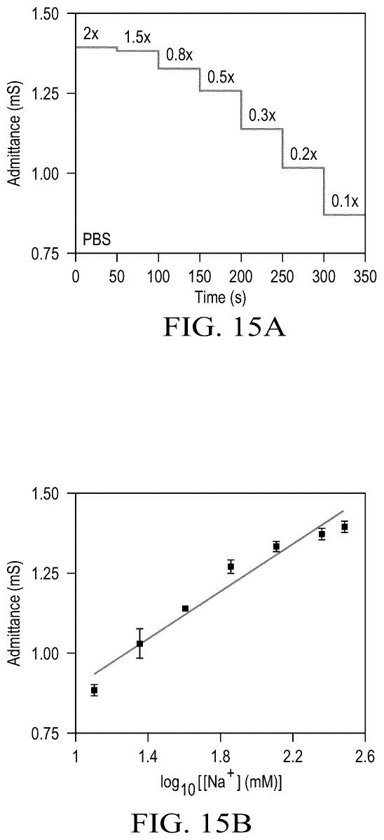

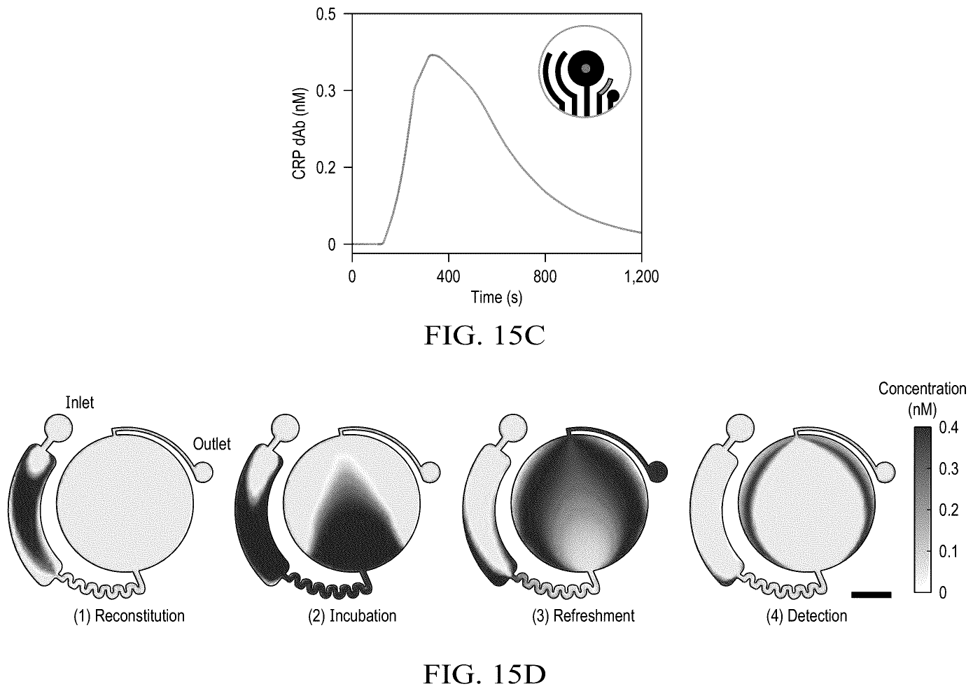

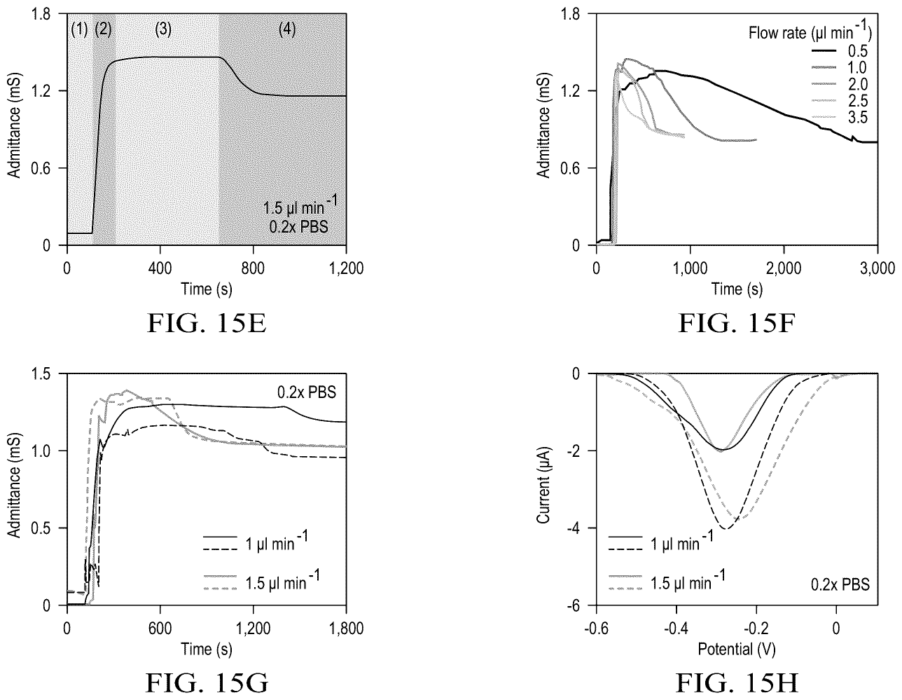

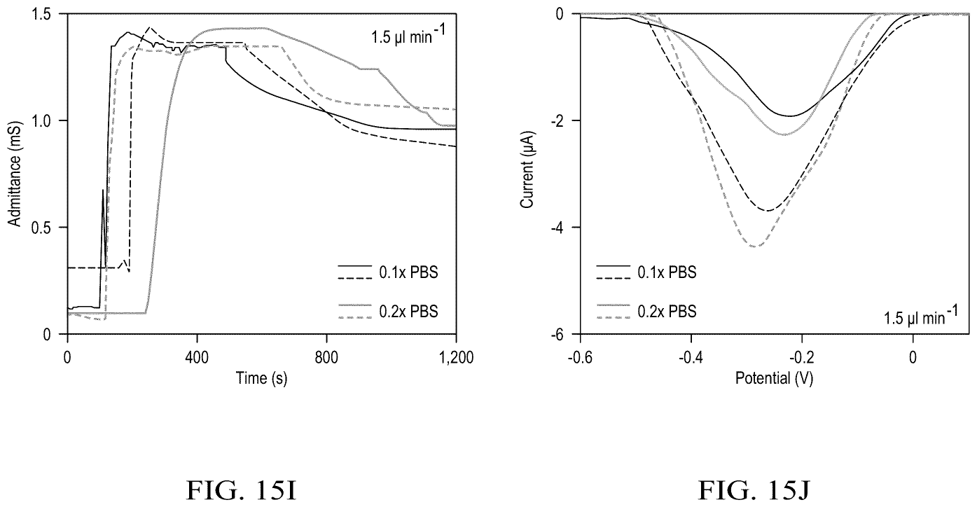

As the microfluidic module routes sweat passively on the skin, the impedimetric ionic strength sensor can automatically capture the state of the detection reservoir (reagent flow and refreshment). FIGS. 15A-15B show measured admittance responses (FIG. 15A) and the corresponding calibration plot (FIG. 15B) of the impedimetric ionic strength sensor in NaCl solutions, where the error bars represent the s.d. of the mean from 3 sensors. As depicted, the measured admittance signals of the impedimetric ionic strength sensor showed a log-linear response with the electrolyte concentrations. As large interpersonal variations in electrolyte and pH levels were observed in both exercise and chemically induced sweat samples, high-level buffering salts were deposited with the dAbs in the reagent reservoir to mitigate potential binding environment changes caused by sweat composition variations. This addition introduced an electrolyte gradient between the detection reagent reconstituted sweat (mixture) and fresh sweat that subsequently entered the detection reservoir. According to a numerical simulation, further described below, the routing of sweat and detection reagents can be summarized into four steps: reconstitution (I), incubation (II), refreshment (III), and detection (IV).

As sweat samples containing CRP molecules enter the microfluidic patch, it was expected that detector antibodies deposited in solid state would dissolve and diffuse within the detection chamber along the concentration gradient. The collision between CRP molecules with antibodies would lead to the antigen-antibody binding events along the microfluidic channels before they eventually reach the detection chamber. The introduction of a serpentine microfluidic channel was also expected to facilitate the mixing and binding of the antigen-antibody complex.

To visualize and estimate the time scale of the binding events at various locations of the microfluidic module, simulation of the CRP-antibody reversible binding reaction and the mass transport process of reactant and product were conducted through finite element analysis (FEA). Using FEA, tetrahedral elements with refined meshes allowed modeling of the source diffusion in 3D space with testified accuracy. The chemical reaction rate can be described by law of mass action

r=k^{f}C_{CRP}·C_{antibody}−k^{r}C_{complex}

Where r, k^{f}, k^{r}, C_{CRP}, C_{antibody}, and C complex denote reaction rate, forward reaction coefficient, reverse reaction coefficient, concentration of CRP, concentration of antibody and concentration of CRP-antibody complex, respectively. The forward and reverse reaction coefficients were assumed to be 5.96×10⁴ M⁻¹s⁻¹ and 2.48×10⁻³ s⁻¹, respectively. The concentration of CRP in sweat was assumed to be 1 ng mL⁻¹. The fluid behavior can be described by the Navier-Stokes equation for incompressible flow

$$ρ ( \frac{\partial v}{\partial t} + ( v \cdot Δ v ) ) = - ∇ p + μ {∇}^{2} v \\ ∇ \cdot v = 0$$

Where ρ, v, p, and μ denote liquid density, flow velocity, pressure, and viscosity, respectively. The sweat flow rate is 1.5 μg mL⁻¹. And the convection diffusion is described by

$$\frac{\partial c}{\partial t} + v \cdot ∇ c = D {∇}^{2} c$$

Where c and D denote concentration and diffusion coefficient. The diffusion coefficient of CRP is 5×10⁻¹¹ m⁻²s⁻¹, the diffusion coefficient of antibody and CRP-antibody complex are set to be the same as gold nanoparticles which is 1×10⁻¹² m⁻²s⁻¹.

FIGS. 15C-15D illustrate results of performing the FEA. FIG. 15C shows simulated CRP-dAb concentration changes on the working electrode over time, where the center dot in the working electrode of the inset image indicates the location of the concentration change plot. FIG. 15D shows simulated CRP-dAb concentrations showing phases of automatic sweat sampling and reagents routing toward in situ CRP detection: reconstitution (I), incubation (II), refreshment (III), and detection (IV). Scale bar, 200 μm.

Based on the observed results, the binding and transport of CRP with detection antibodies can be categorized into four stages. The maps of FIG. 15D represent the concentration of CRP-detection antibody complex formed. In the reconstitution stage, detection antibodies diffuse along the concentration gradient. Binding of CRP starts to occur within the center of the reagent reservoir. As more sweat containing CRP molecules enter the reagent reservoir, more antigen-antibody complexes are formed as illustrated by FIG. 15D. The antigen-antibody complex travels along the flow direction to enter the detection chamber. After the serpentine mixing channels, antigen-antibody complex slowly distributes evenly across the detection chamber, allowing binding with capture antibodies immobilized at the bottom of the detection chamber to occur (incubation stage).

Based on the observed FEA results, after all the pre-deposited detection antibodies in the reagent reservoir are reconstituted, formed antigen-antibody complex with sweat CRP or flushed into the detection reservoir, the concentration of detection antibodies in the reagent reservoir is gradually depleted. The continuous flow of sweat into the microfluidic module will no longer lead to the formation of more antibody-antigen complexes as indicated by concentration in the reagent reservoir during the refreshment stage. Hence, fresh sweat stream deplete of antigen-antibody complexes continues to enter the detection chamber and flush the unbound antibody-antigen complexes in the chamber towards the outlet. Eventually, all unbound antibody-antigen complexes and detection antibodies (which are labeled with electroactive molecules) will be refreshed out of the detection chamber as shown in the detection stage. At this stage, detection is performed, and the electrochemical signal obtained is specific and correlated to the antigen-antibody complexes bound on the working electrode surface since the concentration of the complex in the detection chamber converges to zero (indicated by the concentration).

Based on a microfluidic flow test using artificial sweat (0.2× PBS) under a mean physiological sweat rate (1.5 μL min⁻¹), it was observed that the admittance signal is close to zero initially when no fluid enters the chamber during the reconstitution stage; as reconstituted, high-salt loaded detection reagents flow into the detection chamber, admittance reaches its peak value and gradually decreases as high-salt loaded reagents are flushed out of the detection chamber by newly secreted sweat. This is illustrated by FIG. 15E, which shows admittance changes of the LEG ionic strength sensor as a function of time during the aforementioned four stages of automatic CRP sensing process in a laboratory flow test using artificial sweat (0.2× PBS) at a flow rate of 1.5 μL min⁻¹. In this example flow test, yellow fluorescein isothiocyanate (FITC)-albumin fluorescent label was used to imitate the flow of sweat CRP and red Peridinin Chlorophyll Protein Complex (PerCP) was used in place of dAb-loaded AuNPs. Scale bar, 200 μm. Because electrolyte content in iontophoresis sweat can remain relatively stable for the same individual, the admittance response was observed to plateau after all reagents have been refreshed by natural sweat, indicating the working electrode is ready for electrochemical CRP detection. Further experimental flow tests using the fluorescent proteins (fluorescein isothiocyanate-albumin as CRP surrogate and peridinin chlorophyll protein as detection reagent) showed a similar trend in incubation and refreshment process as the simulation and electrolyte flow test. Based on sweat rate information collected from 24 current and former smokers with and without COPD, flow tests with flow rates varying from 0.5 to 3.5 μL min⁻¹ showed similar admittance patterns with plateaus after various refreshing processes. This is illustrated by FIG. 15F, which shows admittance responses of the ionic strength sensor in artificial sweat (0.2× PBS) at different flow rates from 0.5 to 3.5 μL min⁻¹. The gradient of admittance at different flow rates converges to zero, as pre-loaded salts and dye are refreshed from the detection reservoir. The mean sweat volume routed during this process before sensors readings were taken was estimated to be 21 μL based on flow rate and admittance measurements as shown in FIG. 15F.

The performance of CRP sensors based on this automated electrolyte monitoring mechanism was evaluated in multiple microfluidic flow tests. FIGS. 15G-H are plots illustrating the influence of flow rates on microfluidic automatic CRP sensing. FIGS. 1-J are plots illustrating the influence of ionic strengths on microfluidic automatic CRP sensing. Solid and dotted lines represent tests performed in 1 and 5 ng mL⁻¹ CRP, respectively. SWV electrochemical measurements were initiated during the admittance plateaus. It was observed that an increased concentration (from 1 to 5 ng mL⁻¹) led to an increased SWV peak current height while no substantial difference in CRP sensor response was observed for the same concentration under physiologically relevant flow rates (1, 1.5, 2.5, and 3.5 μL min⁻¹). Although a higher flow rate could also result in a faster refreshment of the detection chamber and thus a shorter incubation time for the detection antibody and CRP, the increment in CRP signals under varying incubation time corresponding to the physiologically relevant sweat rates (between 5 and 20 minutes) was observed to be relatively small.

Although the binding condition is pre-adjusted with deposited salts, the flow test with different initial electrolyte concentrations (0.1× and 0.2× PBS were chosen as artificial sweat to simulate interpersonal variations in sweat electrolyte concentrations) showed slightly decreased SWV signals at the lower electrolyte concentration due to the influence of electrolyte levels on the rate of TH reduction. Similar to in vitro selectivity results, no major interferences on the CRP detection signal were observed in the flow test. Additionally, flow tests using artificial sweat with different pH levels lead to varied SWV signals. These results indicate that sweat rate calibration may not be needed while additional in situ signal calibrations with sweat pH and electrolyte levels may be needed to mitigate the interpersonal variations on CRP detection accuracy. Compared to previously reported passive wearable microfluidic sensors that rely on vigorous exercise to induce sweat and cannot reach sensitivities below mM levels, the technology described herein can an attractive fully automated microfluidic sweat induction, harvesting, and high-accuracy quantitative analysis solution, suitable for at-home monitoring of clinically relevant trace-level biomarkers.

Real-Time CRP Sensor Calibration During On-Body Studies

The influence of pH, electrolyte and temperature were investigated, and all were found to be factors that could influence the sensor readout of CRP. To account for the influences from binding environments, in a particular embodiment a multivariate model consisting of four independent variables: temperature, pH, electrolyte, CRP concentration ([CRP]) and a dependent variable: peak current expressed in potential (mV) was constructed based on the following equation:

peak current=A×[CRP]×pH^{m}×[electrolyte]ⁿ×temperature^{j}

In a particular embodiment, the coefficients were solved using non-linear least square fitting and found to be: A=−0.5117; m=0.6862; n=0.1068; j=−0.6135. The model demonstrated good accuracy in predicting signals measured by the sensors (r²=0.94). During on-body operation, readings from the pH, temperature, electrolyte, and CRP sensors can thus be used to real-time back-calculate the actual concentration of CRP based on the fitted model.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The terms “substantially” and “about” used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

To the extent applicable, the terms “first,” “second,” “third,” etc. herein are merely employed to show the respective objects described by these terms as separate entities and are not meant to connote a sense of chronological order, unless stated explicitly otherwise herein.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The terms “substantially” and “about” used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

To the extent applicable, the terms “first,” “second,” “third,” etc. herein are merely employed to show the respective objects described by these terms as separate entities and are not meant to connote a sense of chronological order, unless stated explicitly otherwise herein.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It should be appreciated that all combinations of the foregoing concepts (provided such concepts are not mutually inconsistent) are contemplated as being part of the inventive subject matter disclosed herein. In particular, all combinations of claimed subject matter appearing in this disclosure are contemplated as being part of the inventive subject matter disclosed herein.

What is claimed is:

1. A wearable biosensor device, comprising:
an iontophoresis module configured to stimulate production of a sweat sample from skin of a user, the sweat sample including biomarkers;
a microfluidic module configured to collect the sweat sample, mix the sweat sample with labeled detection reagents to obtain a mixture including the biomarkers bound to the labeled detection reagents, and route the mixture to a detection reservoir of the microfluidic module; and
a sensor assembly comprising a bioaffinity sensor configured to quantify the biomarkers of the mixture in the detection reservoir to determine a concentration of the biomarkers present in the sweat sample, the bioaffinity sensor comprising an electrode functionalized to bind to the biomarkers of the mixture.

2. The wearable biosensor device of claim 1, wherein:
the labeled detection reagents comprise first nanoparticles conjugated with detection antibodies that bind to the biomarkers; and
a surface of the electrode comprises second nanoparticles conjugated with capture antibodies that bind to the biomarkers.

3. The wearable biosensor device of claim 2, wherein:
the first nano particles and second nanoparticles are gold nanoparticles; and
the biomarkers comprise protein biomarkers or hormone biomarkers.

4. The wearable biosensor device of claim 1, wherein the bioaffinity sensor is configured to quantify the biomarkers of the mixture to determine the concentration with a sensitivity of 1 micromole or less, 100 nanomoles or less, 10 nanomoles or less, 1 nanomole or less, 100 picomoles or less, or 10 picomoles or less.

5. The wearable biosensor device of claim 1, wherein the microfluidic module comprises:
an inlet for collecting the sweat sample;
a reagent reservoir including the labeled detection reagents, the reagent reservoir configured to refresh the sweat sample with the labeled detection reagents;
a mixing channel for mixing the sweat sample refreshed with the labeled detection reagents to form the mixture including the labeled detection reagents bound to the biomarkers;
the detection reservoir for receiving the mixture from the mixing channel; and
an outlet for providing an outflow of the sweat sample from the detection reservoir.

6. The wearable biosensor device of claim 1, wherein the sensor assembly further comprises:
a temperature sensor configured to measure a temperature of the skin;
an ionic strength sensor configured to measure an ionic strength of the sweat sample; and
a pH sensor configured to measure a pH level of the sweat sample, wherein the wearable biosensor device is configured to calibrate readings from the bioaffinity sensor based on measurements made by the temperature sensor, the ionic strength sensor, and the pH sensor.

7. The wearable bio sensor device claim 6, wherein the sensor assembly comprises a multiplexed sensor array fabricated using laser-engraved graphene (LEG), the multiplexed sensor array including the bioaffinity sensor, the temperature sensor, the ionic strength sensor, and the pH sensor.

8. The wearable biosensor device of claim 1, wherein the wearable biosensor device comprises:
a disposable patch including the iontophoresis module, the microfluidic module, and the sensor assembly, the disposable patch comprising an adhesive to directly adhere the disposable patch to the skin; and
a flexible printed circuit board (FPCB) coupled to the patch, the FPCB configured to receive signals from the sensor assembly and power the wearable biosensor device.

9. The wearable biosensor device of claim 8, wherein:
the FPCB is reusable and configured to removably couple to the patch; and the
FPCB comprises a processor configured to perform in situ signal processing of signals received from the sensor assembly, and a wireless communication module configured to wirelessly communicate, in real-time, with a mobile device.

10. A method, comprising:
receiving, via an inlet of a microfluidic module, a sweat sample collected from skin, the sweat sample including protein or hormone biomarkers;
reconstituting, within a reagent reservoir of the microfluidic module, the sweat sample with detection reagents configured to bind with the protein or hormone biomarkers, the detection regents comprising electroactive label molecules;
binding, within a mixing channel of the microfluidic module, the detection reagents with the protein or hormone biomarkers to form a mixture including the protein or hormone biomarkers bound with the detection reagents;
collecting, within a detection reservoir of the microfluidic module, the mixture of the protein or hormone biomarkers bound to the detection reagents, to bind the protein or hormone biomarkers to an electrode of a sensor assembly;
refreshing the microfluidic module with one or more additional sweat samples not containing detection reagents to remove, via an outlet of the microfluidic module, unbound detection reagents; and
estimating a concentration of the protein or hormone biomarkers present in the sweat sample by measuring an amount of the electroactive labels present at a surface of the electrode.

11. The method of claim 10, wherein estimating the concentration of the protein or hormone biomarkers present in the sweat sample, comprises: estimating the concentration of the protein or hormone biomarkers with a sensitivity of 1 micromole or less, 100 nanomoles or less, 10 nanomoles or less, 1 nanomole or less, 100 picomoles or less, or 10 picomoles or less.

12. The method of claim 10, further comprising:
obtaining, using one or more additional sensors of the sensor assembly, one or more additional biophysical sensor measurements comprising a temperature of the skin, a pH level of the sweat sample, or an ionic strength of the sweat sample; and
calibrating, based on the one or more additional biophysical sensor measurements, the estimated concentration of the protein or hormone biomarkers.

13. The method of claim 10, further comprising: prior to receiving the sweat sample via the inlet, inducing, using an iontophoresis module in contact with the skin, the sweat sample.

14. The method of claim 10, wherein:
the protein biomarkers are C-reactive proteins (CRP);
the detection reagents further comprise first nanoparticles conjugated with detection antibodies that bind to the CRP; and
a surface of the electrode comprises second nanoparticles conjugated with capture antibodies that bind to the CRP.

15. The method of claim 14, wherein:
the first nanoparticles and second nanoparticles are gold nanoparticles; and
the electroactive label molecules are redox molecules.

16. A method, comprising:
adhering, to skin of a user, a patch that includes a microfluidic module and sensor assembly;
collecting, in the microfluidic module, a sweat sample obtained from the skin;
mixing, within the microfluidic module, the sweat sample with reagents to obtain a mixture that comprises the reagents bound to protein or hormone biomarkers contained in the sweat sample; and
estimating, from the mixture, using the sensor assembly, a concentration of the protein or hormone biomarkers in the sweat sample.

17. The method of claim 16, further comprising: monitoring, in real-time, based on the concentration of the protein or hormone biomarkers estimated using the sensor assembly, a health condition of the user.

18. The method of claim 17, wherein monitoring, in real-time, the health condition of the user, comprises: comparing the concentration of the protein or hormone biomarkers estimated using the sensor assembly to a threshold to determine a biological condition of the user.

19. The method of claim 17, wherein the health condition comprises: heart disease, chronic obstructive pulmonary disease, inflammatory bowel disease, an active infection, or a past infection.

20. The method of claim 16, further comprising: presenting to the user, in real-time, via a mobile device communicatively coupled to the patch via a wireless communication medium, the concentration of the protein or hormone biomarkers estimated using the sensor assembly.
